(19) United States
(12) Patent Application Publication
(10) Pub. No.: US 2019/0015035 A1
(43) Pub. Date: Jan. 17, 2019

(54) VESTIBULAR TESTING SYSTEMS AND RELATED METHODS

(71) Applicant: MASSACHUSETTS EYE AND EAR INFIRMARY, Boston, MA (US)

(72) Inventors: Daniel Michael MERFELD, Lincoln, MA (US); Yongwoo YI, Dorchester, MA (US)

(73) Assignee: Massachusetts Eye and Ear Infirmary, Boston, MA (US)

(21) Appl. No.: 16/068,485

(22) PCT Filed: Jan. 6, 2017
(86) PCT No.: PCT/US2017/012456

§ 371 Date: Jul. 6, 2018

Related U.S. Application Data
(60) Provisional application No. 62/275,601, filed on Jan. 6, 2016.

(51) Int. Cl. A61B 5/00

(52) U.S. Cl. CPC A61B 5/4023; A61B 5/702; A61B 5/4884

(57) ABSTRACT

Apparatus and methods for estimating a vestibular function of a subject include a motion platform for supporting a subject and an input device configured to receive confidence ratings from the subject. The motion platform is configured to execute one or more motions. The confidence ratings are related to the subject's perception of the one or more motions. The apparatus further includes a processer configured to fit a cumulative distribution function to the confidence ratings, determine a relationship configured to link the cumulative distribution function to an underlying noise distribution, and output parameters associated with the vestibular function based at least in part on the relationship.

### PRIORITY CLAIM

This application claims priority to U.S. Provisional Application 62/275,601, filed on Jan. 6, 2016, the entire content of which is incorporated herein by reference.

### STATEMENT OF GOVERNMENT RIGHTS

This work was supported in part by NIH/NIDCD grant DC04158 and NIH grant R56DC012038. The United States government may have certain rights in the invention.

### TECHNICAL FIELD

This disclosure relates to vestibular testing systems and methods.

### BACKGROUND

The vestibular system of the inner ear enables one to perceive body position and movement. In an effort to assess the integrity of the vestibular system, it is often useful to test its performance. Such tests are often carried out at a vestibular clinic.

Vestibular clinics typically measure reflexive responses like balance or the vestibulo-ocular reflex (VOR) to diagnose a subject's vestibular system. The VOR is one in which the eyes rotate in an attempt to stabilize an image on the retina. Because the magnitude and direction of the eye rotation depend on the signal provided by the vestibular system, observations of eye rotation provide a basis for inferring the state of the vestibular system. Measurements of eye movement are useful for diagnosing some failures of the vestibular system.

Some patients tested in vestibular clinics can report perceptual vestibular problems and test normal on diagnostic tests that assess the VOR. For example, these diagnostic tests may use reflexive vestibular responses and vestibular perception associated with different neural pathways than those tested in the clinics. The tests may measure average VOR metrics such as gain and phase that may fail to diagnose some vestibular problems. Some disorders may include subtle physiological responses that VOR diagnostic tests are unable to measure. For example, VOR tests typically assess responses to motions with relatively large amplitudes, but some diagnoses may require conducting tests having motions with small amplitudes.

### SUMMARY

The present disclosure is related to apparatus and methods for estimating a vestibular function of a subject. In one aspect, the document describes apparatus that include a motion platform for supporting a subject and an input device configured to receive confidence ratings from the subject. The motion platform is configured to execute one or more motions. The confidence ratings are related to the subject's perception of the one or more motions. The apparatus further include a processer configured to fit a cumulative distribution function to the confidence ratings, determine a relationship configured to link the cumulative distribution function to an underlying noise distribution, and output parameters associated with the vestibular function based at least in part on the relationship. The parameters also provide an estimation of the vestibular function of the subject.

In another aspect, this document features methods for estimating a vestibular function of a subject. The methods include providing one or more motion stimuli to a subject. The methods further include receiving confidence ratings from the subject and fitting a cumulative distribution function to the confidence ratings. The confidence ratings indicate the subject's perceptions of the motion stimuli. The methods further include determining a relationship configured to link the cumulative distributive function to an underlying noise distribution and generating parameters associated with the vestibular function based at least in part on the relationship. The parameters also provide an estimation of the vestibular function of the subject.

In a further aspect, one or more machine-readable storage devices have encoded thereon computer readable instructions for causing one or more processors to perform operations as described herein. The operations include providing one or more motion stimuli to a subject and receiving confidence ratings from the subject. The confidence ratings indicate the subject's perceptions of the motion stimuli. The operations further include fitting a cumulative distribution function to the confidence ratings, determining a scaling parameter configured to link the cumulative distributive function to an underlying noise distribution, and generating parameters associated with the vestibular function. The parameters include the scaling parameter. The parameters also provide an estimation of the vestibular function of the subject.

Implementations of the above aspects can include one or more of the following features. The relationship can be represented by a scaling parameter configured to link the cumulative distribution function associated with the confidence ratings to a cumulative distribution function associated with the underlying noise distribution. The plurality of parameters can include the scaling parameter. The cumulative distribution function associated with the confidence ratings and the cumulative distribution function associated with the underlying noise distribution can both be Gaussian. The cumulative distribution function can be fitted to the confidence ratings using a maximum likelihood criterion.

The cumulative distribution associated with the confidence ratings can be different from a cumulative distribution function associated with the underlying noise distribution.

In some examples, the parameters can include a width and bias of the vestibular function.

In some examples, the confidence ratings include any of a quasi-continuous rating, a binary rating, an N-level discrete rating, or a wagering rating.

The technologies described herein can provide several advantages. For example, the time required to test a subject can be reduced. In particular, the use of the confidence ratings can be used to account for the underlying noise distribution, which can in turn be used to reduce the number of overall trials needed to determine the parameters associated with the vestibular function for a given subject. The additional consideration of the confidence ratings can also decrease the variability of the parameters to provide more precise estimations of the parameters associated with the vestibular function of the subject.

Unless otherwise defined, all technical and scientific terms used herein have the same meaning as commonly understood by one of ordinary skill in the art to which the subject matter of this disclosure belongs. Although methods and materials similar or equivalent to those described herein can be used in the practice or testing of the implementations described herein, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages will be apparent from the following detailed description, and from the claims.

### DESCRIPTION OF DRAWINGS

FIGS. 1A to 1D are graphs depicting an analytic relationship among decision variables, psychometric functions, and confidence functions.

FIGS. 1E to 1H are graphs depicting simulation results representing a relationship among decision variables, psychometric functions, and confidence functions.

FIGS. 2A and 2B are graphs depicting a relationship between confidence probability judgments and a maximum likelihood psychometric function fit.

FIGS. 3A to 3C are graphs depicting example fits for a human test.

FIGS. 4A to 4L are graphs depicting human psychometric parameter estimates.

FIG. 5 is a set of graphs depicting standard deviation of human psychometric parameter estimates.

FIGS. 6A-6F are each a set of graphs depicting parameter estimates for 10,000 simulated experiments with 20 and 100 trials.

FIGS. 7A-7L is a set of graphs depicting simulation parameter estimates.

FIG. 8 is a set of graphs depicting standard deviation of simulation parameter estimates.

FIG. 9 is a set of graphs depicting human psychometric width parameter, confidence scaling factor, and bias parameter estimates.

FIGS. 10A-10D are each a set of graphs depicting parameter distributions.

FIGS. 11A and 11B show flow charts of confidence fit processes.

FIG. 12 is a set of graphs that shows confidence probability judgment distributions for different subjects at different stimulus levels.

FIGS. 13A-13C are graphs that show results of a confidence probability judgment test associated with a fixed-duration direction-recognition task.

FIG. 14 is a schematic of a vestibular testing system.

FIGS. 15A to 15C are schematics showing examples of head orientations along with corresponding head coordinates and earth coordinates.

FIGS. 16A and 16B are schematics showing examples of input devices.

FIG. 17 is a block diagram of a computing system.

Like reference symbols in the various drawings indicate like elements.

### DETAILED DESCRIPTION

Perceptual thresholds are commonly assayed in the lab and clinic. When precision and accuracy are required, thresholds are quantified by fitting a psychometric function to forced-choice data. However, this approach can require a hundred trials or more to yield accurate (i.e., small bias) and precise (i.e., small variance) psychometric parameter estimates. The present disclosure demonstrates that confidence probability judgments combined with a model of confidence can yield psychometric parameter estimates that are markedly more precise and/or markedly more efficient than methods using a signal detection model without consideration of confidence (e.g., confidence-agnostic methods). Specifically, both human data and simulations show that including confidence probability judgments for as few as twenty trials can yield psychometric parameter estimates that match the precision of those obtained from the hundred trials using confidence-agnostic analyses. Such an efficiency advantages are especially beneficial for tasks (e.g., taste, smell, and vestibular assays) that require more than a few seconds for each trial, but the benefits would also accrue for many other tasks.

Measuring thresholds is a psychophysical procedure; applications range from experimental psychology to neuroscience to economics to engineering. Fitting psychometric functions using categorical data analyses that describe the relationship between a stimulus characteristic (e.g., amplitude) and a subject's forced-choice categorical responses provides a standard approach used to estimate thresholds. A comprehensive analysis concluded that maximum likelihood methods can be used when accuracy and precision of psychometric function fit parameters is important and, further, showed that more than a hundred forced-choice trials can be required to yield acceptable fit parameter estimates. Because many trials can be needed to yield accurate and precise psychometric fits, studies spanning fifty years have reported efforts to improve threshold test efficiency (i.e., to reduce the number of trials), but only modest efficiency improvements have accumulated. This may be due to binary/binomial distributions, which can have high variability at near-threshold stimulus levels—where the maximal information can be attained on each trial.

While forced choice procedures can be simple and robust, subjects can know how confident they are for each response. “Confidence” as used herein is a belief in the validity of what a subject believes and is widely considered a form of metacognition, because it involves self-monitoring of perceptual performance. In other words, confidence reflects self-assessment of the conviction in a decision of a subject being tested.

Confidence has been studied in humans using a variety of techniques including probability judgments. In fact, confidence probability judgments (i.e., confidence ratings provided using a nearly continuous scale between 0 and 100% or 50% and 100%) can provide the most common assessment of confidence.

One use of confidence recordings is in “confidence calibration” studies where confidence is compared to actual performance, where a data set may be classified as “well-calibrated” or classified as indicative of “overconfidence” or “underconfidence.” Specifically, assuming that a subject reported 90% confidence that a given motion was rightward for 10 separate trials at a given stimulus level, on average, perfect calibration of these confidence reports is assumed when 9 out of 10 of these trials are in the rightward direction, while overconfidence would be indicated by 5 out of 10 being rightward.

Probability judgments are not typically directly used to help estimate psychometric function parameters. Typically, confidence is not recorded. A confidence rating (e.g., “uncertain”) can be recorded and used as part of a psychometric fit procedure, but these approaches do not model how confidence quantitatively changes as the stimulus is varied. Instead these approaches can include one additional decision boundary for each added category (e.g., “uncertain”)—and can add one free parameter to the fit algorithm for each additional decision boundary.

### Confidence Signal Detection Model

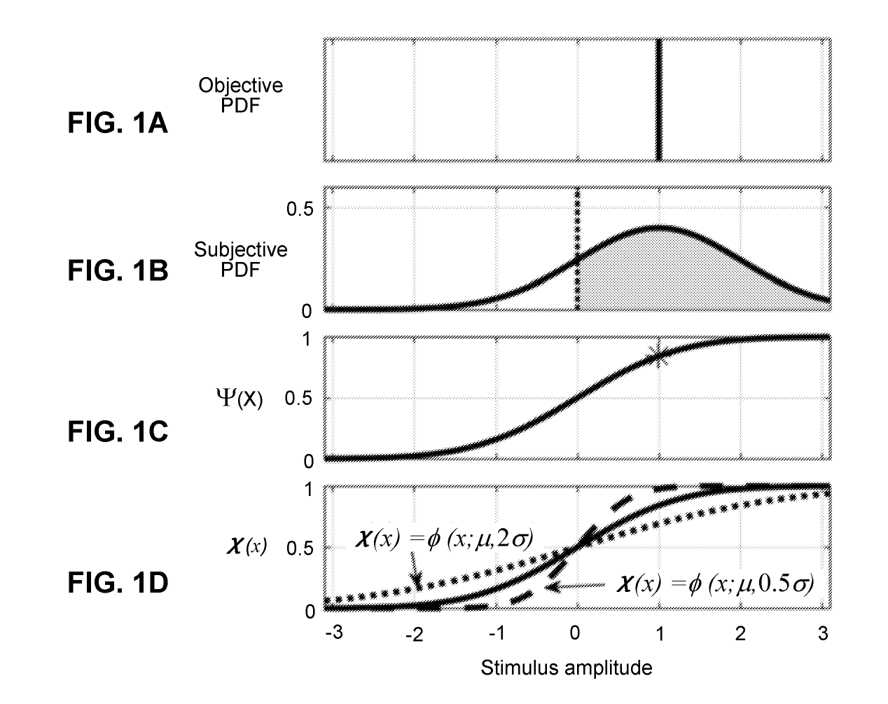

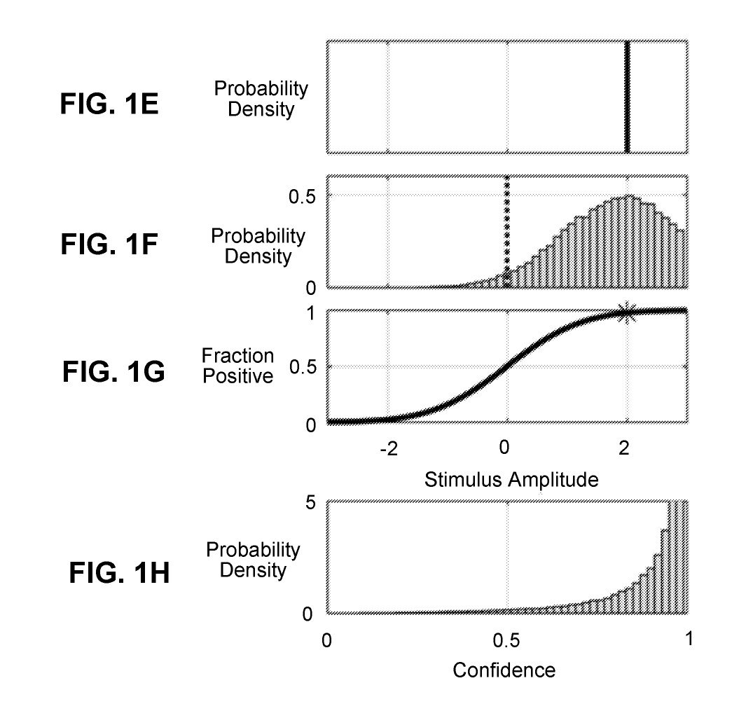

We describe herein a confidence signal detection (CSD) model, which combines a confidence function (FIG. 1D) with a signal detection model (FIGS. 1A-1C).

FIGS. 1 to 10 depict the CSD model and data collected using the CSD model.

FIGS. 1A to 1D depict a relationship between decision variables, psychometric functions (Ψ(x)), and confidence functions (χ(x)) in the confidence signal detection (CSD) model. FIG. 1A shows that the stimulus for this example is well controlled having an amplitude of +1.0 with little variation, so the objective probability density function (PDF) is a delta function. FIG. 1B shows a signal detection model that assumes additive noise. For this example, Gaussian noise having zero-mean and a standard deviation of 1 is added to the stimulus of +1.0 and leads to the subjective PDF shown. The dotted vertical line at zero represents a decision boundary. If a sampled decision variable falls to the right of the decision boundary, represented by the gray area, the subject decides positive. If the sampled decision variable falls to the left, the subject decides negative. For this example, 84% of the decision variables lead to the subject deciding positive. In FIG. 1C, the asterisk, located at (1, 0.84) represents the example data point illustrated in the previous panel. When this process is repeated for a variety of different stimulus levels, it yields a psychometric function, Ψ(x) (black curve). In FIG. 1D, similarly, a relationship between confidence and the stimulus for an individual trial can be represented by a confidence function (χ(x)). The psychometric function represents average subject performance, and confidence is defined as well-calibrated when confidence matches average subject performance. Therefore, well-calibrated confidence matches the psychometric function (χ(x)=Ψ(x)) and is plotted as the solid curve. Also shown are confidence functions that represent over-confidence (dashed) and under-confidence (dotted).

FIGS. 1E-1H illustrate the CSD model using simulations. For the simulations, the stimulus (represented by a delta function) was assumed to have an amplitude equal to 2.0, as shown in FIG. 1E. The physiologic noise (ε) was assumed to be Gaussian with a standard deviation of 1 (σ=1) and mean of zero (μ=0). FIG. 1F shows the simulated distribution of the decision variable across 10,000 trials for the stimulus. Each of these sampled values represents the decision variable available to the nervous system for a single trial for the given noise distribution and the given stimulus level. Assuming no decision biases i.e., that the a priori probability for the direction of each stimulus (e.g., left or right), as well as the costs for all decisions are equal—the ideal signal detector would set a decision boundary at zero. The decision boundary represents the border that delineates whether the subject decides positive or negative. Having the decision boundary at zero represents that when an individual trial yields a positive decision-variable, the subject reports positive (“right”), and when an individual trial yields a negative decision-variable, the subject reports negative (“left”). For the example shown, the predicted distribution falls above the decision boundary (“right”) 97.7% of the time and below the decision boundary (“left”) 2.3% of the time. This 97.7% data point is shown using a cross symbol at the stimulus level of 2.0 in FIG. 1G.

When this process is repeated many times for many different stimulus levels controlled by an operator, a psychometric dataset is generated, which can be quantified by fitting a psychometric function to the dataset. Such a psychometric function can reflect an expected average performance at each stimulus level. For a Gaussian noise distribution, the psychometric function is a Gaussian cumulative density function, as shown in FIG. 1G. Such a fitted Gaussian cumulative distribution function (φ) can be parameterized using two fit parameters \hat{μ}, \hat{σ}, and therefore be represented as \hat{Ψ}(x)=ϕ(x:\hat{μ}, \hat{σ}). The points on the cumulative distribution function can also be determined empirically by repeating the process described above for stimulus levels other than 2. With sufficient amount of data, the empirically determined psychometric function can converge to a function representative of the underlying noise distribution representing the vestibular noise of the subject, which may be represented as \hat{Ψ}(x)≈\hat{Ψ}(x).

A relationship between a confidence function and the psychometric function can also be determined. For a well-calibrated subject performing a symmetric task (i.e., a task having uniform priors), a perfect confidence calibration can be defined to occur when subjective confidence matches objectively assessed accuracy. For example, if a subject reported with 90% confidence that a given motion was rightward for ten separate trials at a given rightward stimulus level, perfect calibration would be reflected, on average, by 9 out of 10 of these trials actually being in the rightward direction. Because average decision-making performance is represented by the psychometric function, a perfectly calibrated confidence is reflected by a confidence function that is substantially identical to the psychometric function, i.e., χ(x)=Ψ(x)=φ(x, μ, σ). For example, for a given trial, consider that a well-calibrated subject has sampled a decision variable having an amplitude of 2.0 (i.e., one trial from the distribution shown in FIG. 1F) that he/she wishes to convert to a confidence probability judgment.

Assuming that the subject has an accurate estimate of the noise distribution (σ=1 and μ=0) and ignoring the effect of any underlying neural processes, a well calibrated subject calculates the confidence by directly mapping the decision variable for each trial onto a respective “confidence function” to determine a confidence probability judgment, which for this trial yields 97.7%, i.e., φ(x=2, μ=0, σ=1)=0.977. When confidence is similarly calculated for each sampled decision variable (each of the trials represented in FIG. 1F), this process yields one confidence value for each sampled decision variable. For this simulation, these confidence values yielded the normalized confidence histogram shown in FIG. 1H.

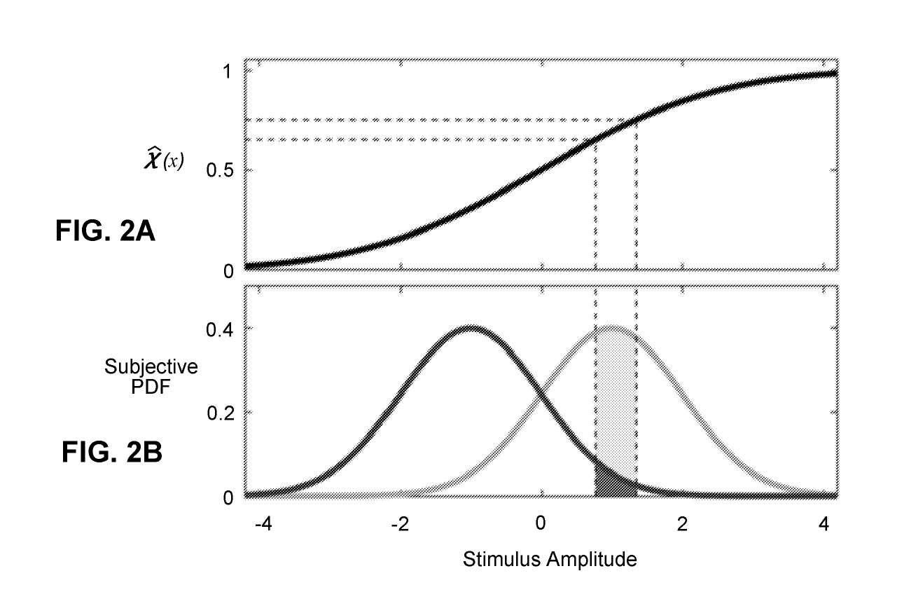

FIGS. 2A to 2B illustrate how confidence probability judgments from individual trials contribute to a maximum likelihood psychometric function fit. As shown in FIG. 2A, given a confidence probability judgment, we can use the inverse fitted confidence function (\hat{χ}⁻¹(cⱼ)) to calculate a modeled decision variable to accompany that judgment. More specifically, given upper and lower limits to a confidence probability judgment (dashed horizontal lines), we can use the inverse fitted confidence function to calculate the corresponding upper and lower decision variable limits (dashed vertical lines). As shown in FIG. 2B, given the estimated decision variable range shown by the dashed vertical lines, we can calculate the probability that the given stimulus (sⱼ) and psychometric function noise model would yield that confidence probability judgment. Two examples are illustrated. The light curve shows the decision variable PDF for the stimulus having an amplitude of +1.0 shown in FIG. 1B and the light shaded area represents the probability of the confidence probability judgment for a +1.0 stimulus. The dark curve shows the PDF for a stimulus having an amplitude of −1.0, and the dark shaded area represents the probability for a −1.0 stimulus. High confidence that the motion is positive is much more probable (i.e., much more likely) for the +1 stimulus than for the −1 stimulus.

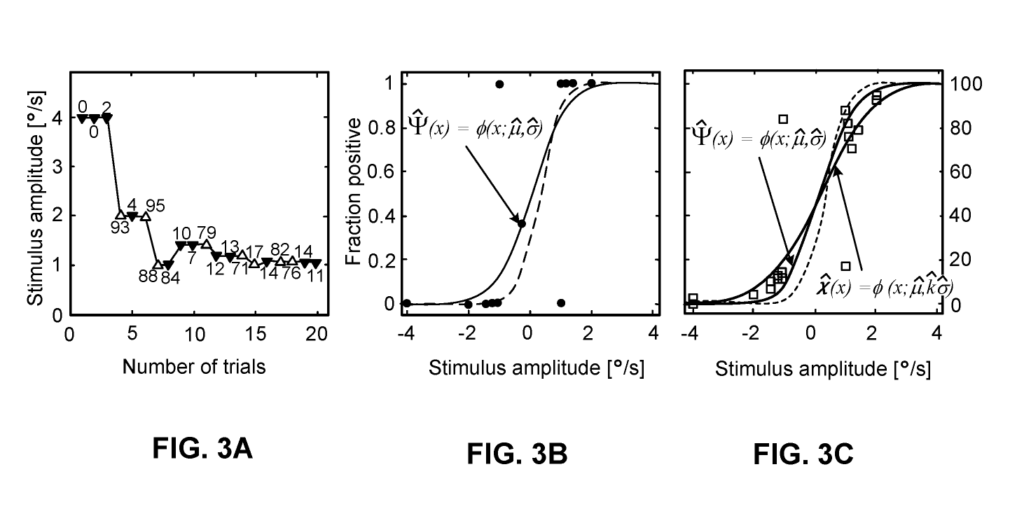

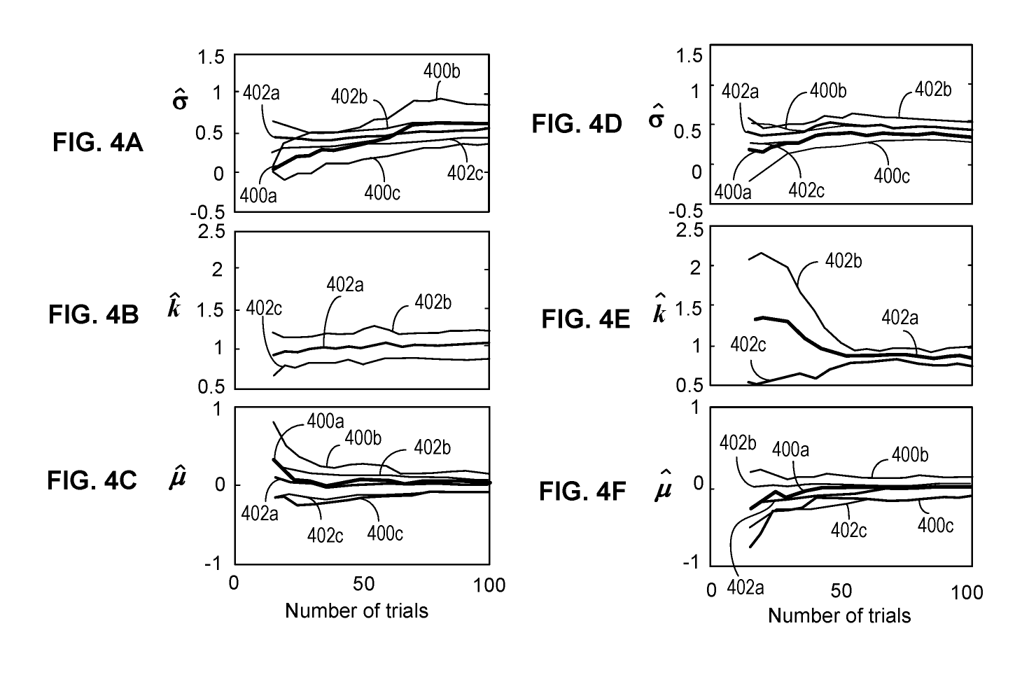

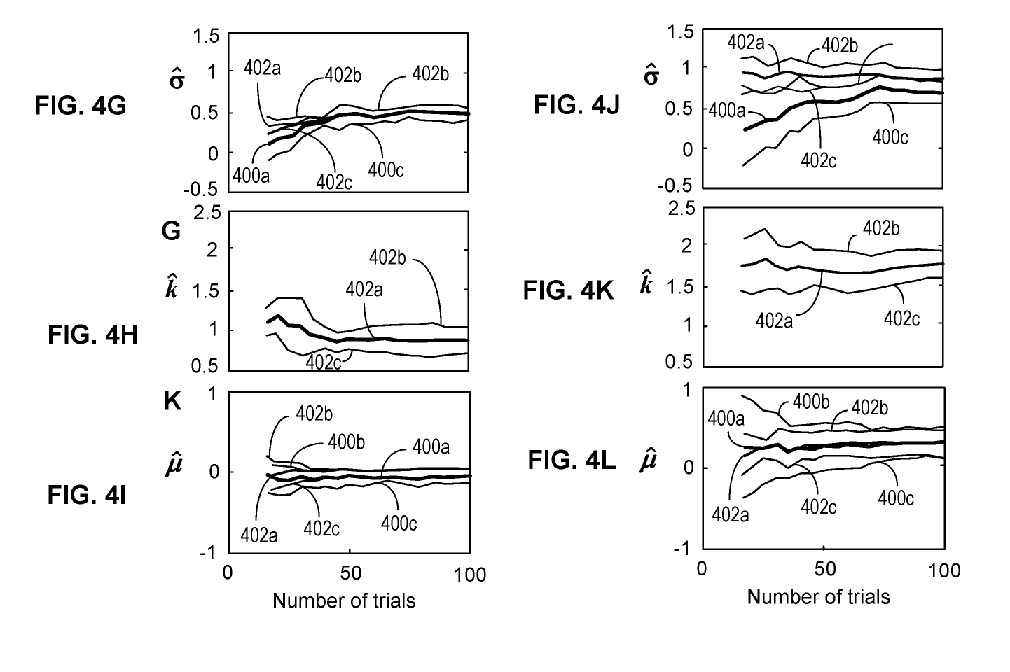

FIGS. 3A to 3C shows example fits for a human test. FIG. 3A shows an example stimulus track, including confidence probability judgments, for the first twenty trials. Upward-pointing gray triangles and downward-pointing black triangles represent rightward and leftward trials, respectively. As shown in FIG. 3B, following twenty binary forced-choice trials, a confidence-agnostic psychometric function (black curve), \hat{Ψ}(x)=ϕ(x; \hat{μ}=0.05,\hat{σ}=0.95), was fit to the binary forced-choice data points shown. As shown in FIG. 3C, given the same twenty trials with confidence probability judgments, a psychometric function (black curve), \hat{Ψ}(x)=ϕ(x; \hat{μ}=0.19,\hat{σ}=0.91), and a confidence function (gray curve), \hat{χ}(x)=ϕ(x; \hat{μ}=0.19, \hat{k}\hat{σ}=1.43), were simultaneously fit to the confidence data. All example data are from one of the human data sets (FIGS. 4A-4C) presented herein. For comparison, the fitted psychometric function determined after a hundred binary forced-choice trials using confidence-agnostic methods, \hat{Ψ}(x)=ϕ(x; \hat{μ}=0.33, \hat{σ}=0.59), is also shown via dashed lines on panels b and c. Half-scale (50% to 100%) probability judgments provided by subjects have been converted to full-scale (0 to 100%) judgments as described in Methods.

FIGS. 4A to 4L show a summary of human psychometric parameter estimates as trial number increases. Each column represents fitted parameters for one subject. FIGS. 4A, 4D, 4G, and 4J show average fitted psychometric width parameter (\hat{σ}). FIGS. 4B, 4E, 4H, and 4K show average fitted confidence scaling factor (\hat{k}). FIGS. 4C, 4F, 4I, and 4L show average fitted psychometric function bias (\hat{μ}) Curves 400a show average psychometric parameter estimates calculated using confidence-agnostic forced-choice analyses. Curves 402a show average parameter estimates determined by fitting confidence probability judgment data. Errors bars (curves 400b, 400c for curves 400a and curves 402b, 402c for curves 402a) represent standard deviation of parameter estimates.

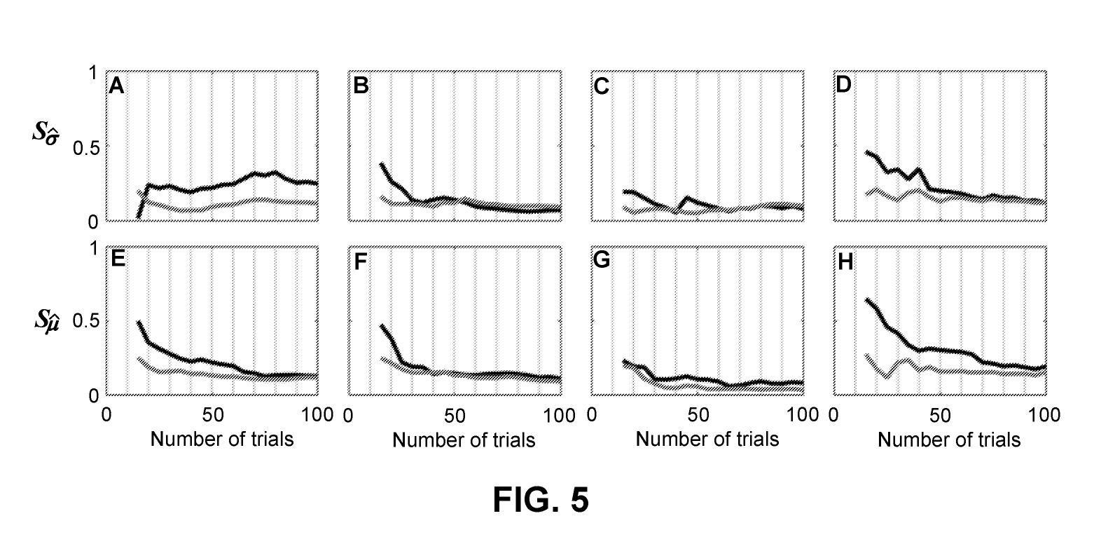

FIG. 5 shows standard deviation of human psychometric parameter estimates as trial number increases. Each column represents fitted parameters for one subject in the same order as FIGS. 4A to 4L. Top row (Panels A-D) represents the standard deviation of the fitted psychometric width parameter (\hat{σ}). Bottom row (Panels E-H) represents the fitted psychometric function bias (\hat{μ}). Black curves show standard deviation of psychometric parameter estimates calculated using confidence-agnostic forced-choice analyses. Gray curves show standard deviation of parameter estimates determined via the CSD model fit.

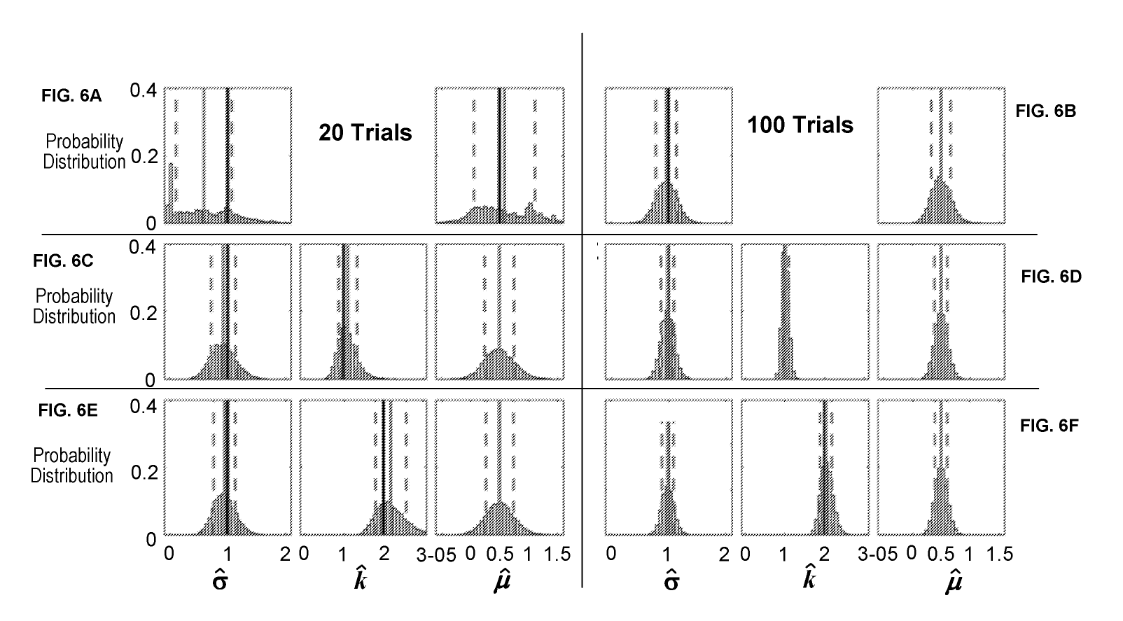

In FIGS. 6A-6F, parameter distributions show parameter estimates for 10,000 simulated experiments with 20 and 100 trials. The columns from left to right represent the fitted psychometric width parameter (\hat{σ}), the fitted confidence scaling factor (\hat{k}) and the fitted psychometric function bias (\hat{μ}) as shown on the x-axis at bottom. Top row (FIGS. 6A and 6B) represents fitted parameters of confidence-agnostic binary forced-choice parameter estimates. Middle row (FIGS. 6C and 6D) represents fitted parameters estimates determined via the CSD model fit for a well-calibrated subject (k=1). Bottom row (FIGS. 6E and 6F) represents fitted parameters estimates determined via the CSD model fit for an under-confident subject (k=2). The solid black line shows the actual parameter value (i.e., μ=0.5 or σ=1), the solid gray line shows the mean of fitted parameters, and the dashed gray lines indicate standard deviation on each side of the mean.

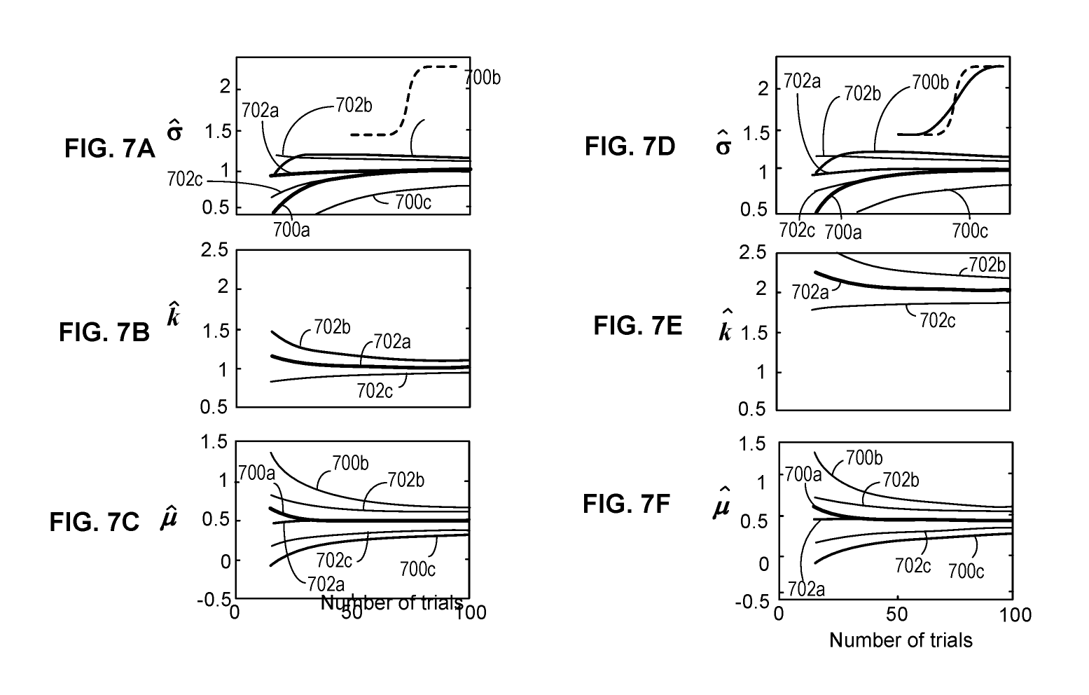

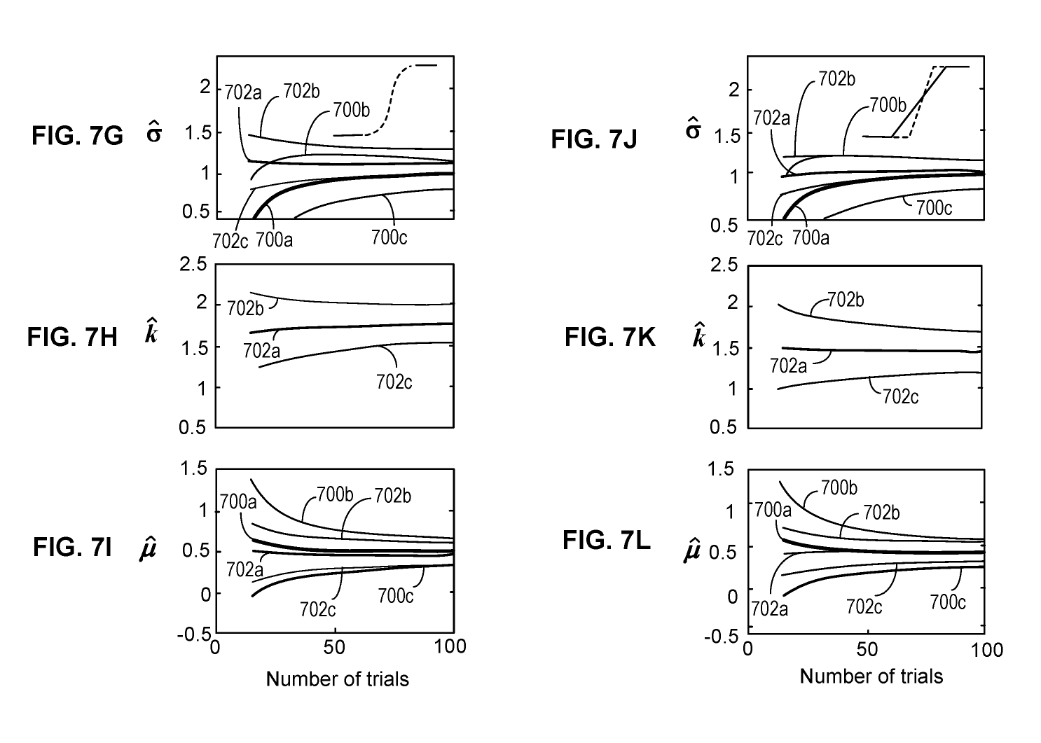

FIGS. 7A-7L show a summary of simulation parameter estimates as trial number increases. Each column represents different simulated combinations of the confidence function (red solid curves) and the fitted confidence function (red dashed curves). FIGS. 7A-7C show a well-calibrated subject (k=1) when both confidence and confidence fit functions are cumulative Gaussians. FIGS. 7D-7F show an under-confident subject (k=2) when both confidence and confidence fit functions are cumulative Gaussians. FIGS. 7G-7I show an under-confident subject when the confidence function is linear, χ(x)=m(x−μ)+0.5=0.1443x+0.428, with added zero-mean uniform noise (U(−0.1, +0.1)), and the confidence fit function is a cumulative Gaussian. FIGS. 7J-7L show an under-confident subject with the same linear confidence function with added zero-mean uniform noise (U(−0.05, +0.05)) when the confidence fit function is linear, \hat{χ}(x)=\hat{m}(x−\hat{μ})+0.5. FIGS. 7A, 7D, 7G, and 7J show fitted psychometric width parameter (\hat{σ}). FIGS. 7B, 7E, 7H, and 7K show fitted confidence-scaling factor (k) or (H) fitted slope of confidence function. FIGS. 7C, 7F, 7I, and 7L show fitted psychometric function bias (\hat{μ}). Curves 700a show average confidence-agnostic forced-choice parameter estimates, which are identical for all conditions. Curves 702a show average parameter estimates determined by fitting confidence probability judgments. Errors bars (curves 700a, 700a for curves 700a and curves 702b, 702c for curves 702a) represent standard deviation of parameter estimates.

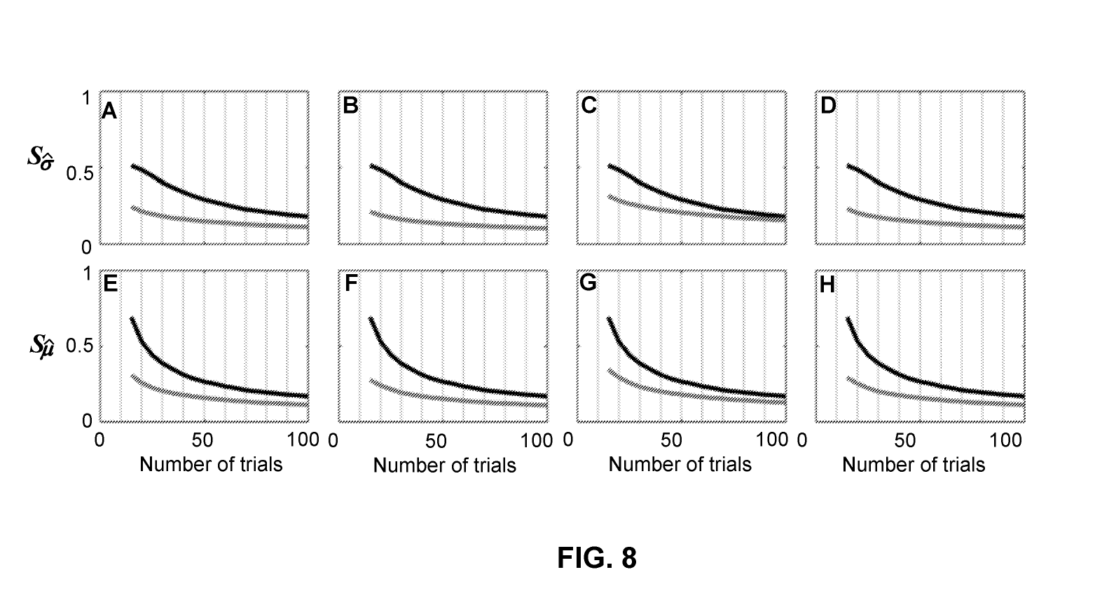

FIG. 8 shows standard deviation of simulation parameter estimates as trial number increases. Each column represents the same conditions as in FIGS. 7A-7C. Top row (Panels A-D) represents the fitted psychometric width parameter (\hat{σ}). Bottom row (Panels E-G) represents the fitted psychometric function bias (\hat{μ}). Black curves show standard deviation of confidence-agnostic forced-choice parameter estimates, which are identical for all conditions. Gray curves show standard deviation of parameter estimates determined via the CSD model fit.

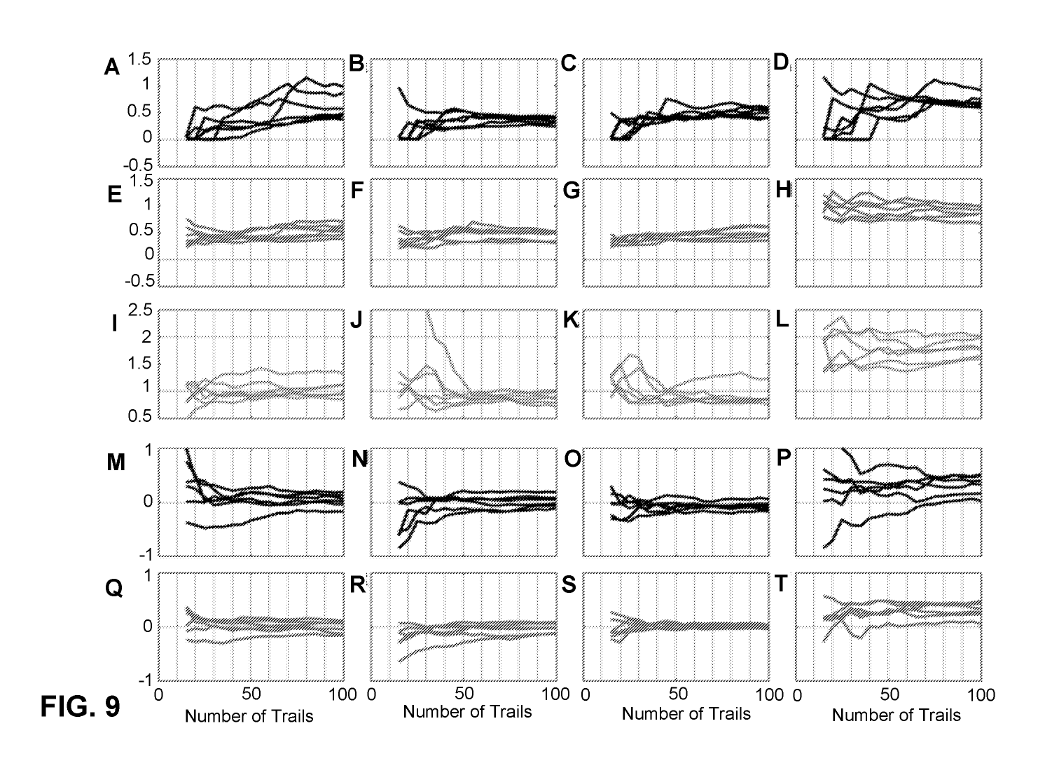

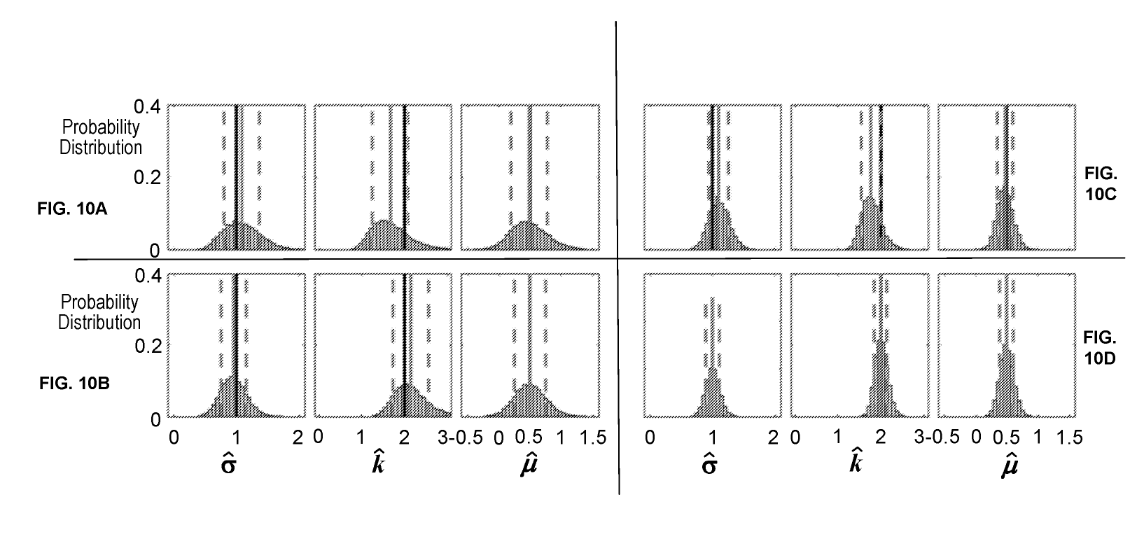

FIG. 9 shows human psychometric width parameter (\hat{σ}), confidence scaling factor (\hat{k}) and, bias parameter (\hat{μ}) estimates as trial number increases for each subject for each of 6 test sessions. Each column shows fitted parameters for one subject. Top row (Panels A-D) shows fitted psychometric width parameter using confidence-agnostic forced-choice analyses. Second row (Panels E-H) shows fitted psychometric width parameter for CSD model fit. Third row (Panels I-L) shows fitted confidence scaling factor for CSD model fit. Fourth row (Panels M-P) shows fitted psychometric function bias using confidence-agnostic forced-choice analyses. Bottom row (Q-T) shows fitted psychometric function bias for CSD model fit.

In FIGS. 10A-10D, parameter distributions show parameter estimates for 10,000 simulated experiments with 20 and 100 trials. Top row (FIGS. 10A and 10C) represents fitted parameters estimates determined via the CSD model fit for an under-confident subject when the confidence function is linear, χ(x)=m(x−μ)+0.5=0.1443x+0.428, with added zero-mean uniform noise (U(−0.1, +0.1)), and the confidence fit function is a cumulative Gaussian. Bottom row (FIGS. 10B and 10D) represents fitted parameters estimates determined via the CSD model fit for an under-confident subject with the same linear confidence function with added zero-mean uniform noise (U(−0.05, +0.05)) when the confidence fit function is linear, \hat{χ}(x)=\hat{m}(x−\hat{μ})+0.5. The solid black line shows the actual parameter value (i.e., μ=0.5 or σ=1), the solid gray line shows the mean of fitted parameters, and the dashed gray lines indicate standard deviation on each side of the mean.

We use an example to illustrate the relationship between psychometric functions and confidence for a direction-recognition forced-choice task. A typical perceptual direction-recognition paradigm begins with well-controlled stimuli that are either positive or negative; the subject's task is to determine whether the motion is positive (“rightward”) or negative (“leftward”). The stimuli provided to a subject (FIG. 1A) can be well controlled (i.e., have little variation). The standard signal detection model suggests that neural noise contributes to perception, which is represented by the probability density function (PDF) shown in FIG. 1B. Signal detection theory advocates that a single sample from this probability distribution—often called the decision variable—is available to the subject for each trial. If the decision variable sampled from this PDF for an individual trial is negative, the subject reports negative (e.g., leftward) motion and if the sampled decision variable is positive, the subject reports positive (e.g., rightward) motion. For the stimulus and noise PDF shown, positive motion will, on average, be reported 84% of the time. When this process is repeated for different stimulus amplitudes, it leads to a fitted psychometric function, (\hat{ψ}(x)), that represents subject performance as a function of stimulus amplitude (FIG. 1C). Large positive stimuli can be correctly reported as positive, and large negative stimuli can be correctly reported as negative. Stimuli in between lead to the sigmoidal shape shown. With enough data, this fitted psychometric function (\hat{ψ}(x)) can converge to a psychometric function that is representative of the subject's underlying noise distribution, \hat{ψ}(x)≈ψ(x).

We now add a confidence model to the above-described signal detection approach. If we sample a very positive decision variable for one trial (e.g., if the stimuli were very large), we can assume high confidence that the motion was positive. If we sample a positive decision variable near the decision boundary on another trial, we can decide that the motion was positive but have much less confidence in that decision. Like the empiric relationship captured by a psychometric function ((\hat{ψ}(x)), a quantitative empiric relationship between confidence and the stimulus can be represented by a confidence function (\hat{χ}(x)). As for the psychometric function, with enough data, this empiric confidence function can be assumed to be representative of neural processes that can be captured by a confidence function, \hat{χ}(x)≈χ(x). FIG. 1D shows three example confidence functions that are each modeled as Gaussian cumulative distribution functions (CDFs). The solid curve represents well-calibrated confidence (χ(x)=ψ(x)), the dashed curve represents over-confidence, and the dotted curve represents under-confidence.

As described in detail in the Methods section entitled “Confidence Maximum Likelihood Fit Technique,” we utilize this CSD model to help improve psychometric parameter estimates. More specifically, we present a confidence analysis technique that utilizes this CSD model. We describe, develop, and investigate this model using previously published analytic, simulation, and experimental approaches. To help evaluate the contributions that confidence can make to psychometric function estimation, we report: (1) human studies for a direction-recognition task in which the subjects were required to report whether they rotated toward their left or right, and (2) simulation results for psychometric functions that range from 0 to 1, which are used for direction-recognition data analysis. We report that psychometric functions estimated using confidence probability judgments can require about 5 times fewer trials to yield the same performance as forced-choice psychometric methods without confidence analysis.

Such improved test efficiency should be realized for any forced-choice task where confidence can be reported, but may be especially important for perceptual tasks involving olfaction, gustation, equilibrium, or any other task where individual trials take, for example, tens of seconds as well as for clinical applications where more efficient and/or more precise perceptual measures could lead to improved patient diagnoses.

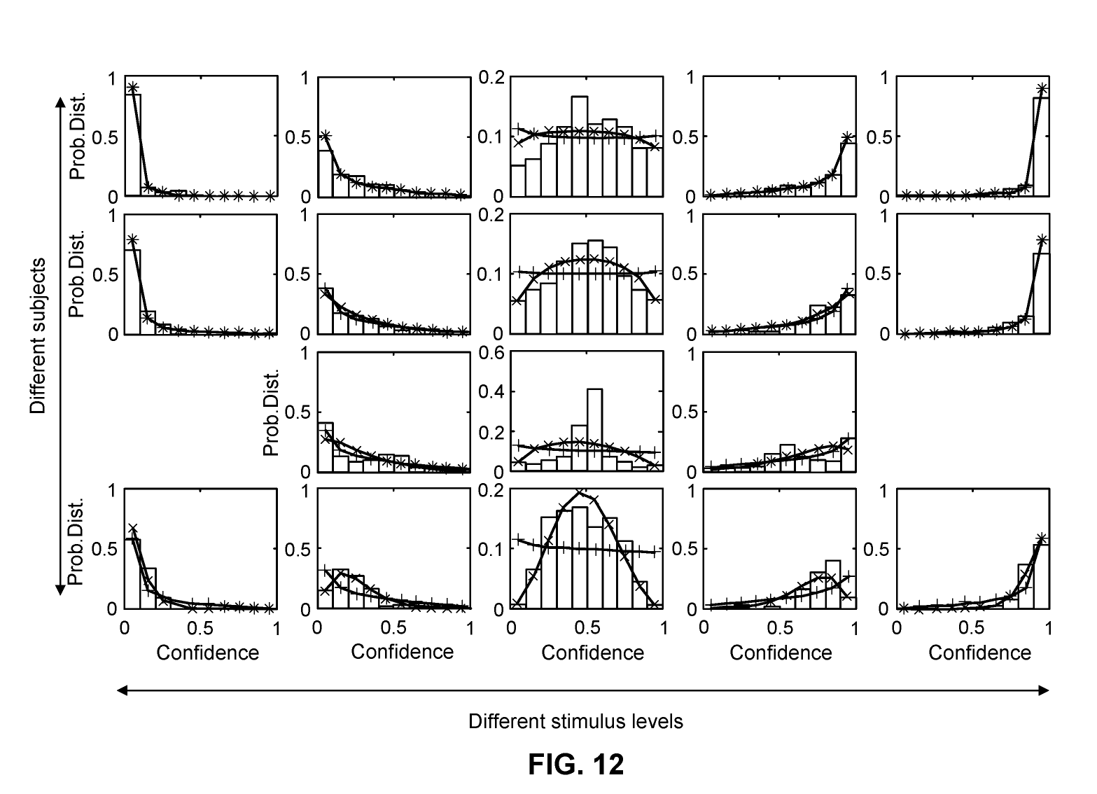

In some implementations, the CSD model can be used to analyze confidence probability judgments. This is illustrated using FIG. 12, which shows the results of an experiment where confidence distributions for four human subjects were empirically determined. Specifically, FIG. 12 shows confidence probability judgment distributions for the different subjects at different stimulus levels. For these experiments, a non-adaptive sampling scheme was used, to allow for repeated trials at different stimulus levels. Each row of plots in FIG. 12 represents one of the four subjects (ordered from the subject S1 with the lowest confidence scaling factor at the top to the subject S4 with the highest confidence scaling factor at the bottom). The histograms in each plot show empirical human data at each of the five stimulus levels, wherein each column represents a different stimulus level. The largest stimulus magnitudes are represented by the first and fifth columns, the second largest stimulus magnitudes are represented by the second and the fourth columns, and the smallest stimulus is represented by the third column. For the subject S3, because the stimuli tested were smaller relative to the actual threshold than for the other three subjects, the second and fourth column show the largest stimulus magnitudes, and the third column shows the confidence for the remaining subthreshold stimuli. The actual stimulus levels (in peak stimulus velocity in degrees per second) used for each subject are provided below in Table 1.

TABLE 1
Experimental stimulus amplitude

| | | | | | | |
|---|---|---|---|---|---|---|
| S1 | ±0.03* | ±0.10 | ±0.20 | ±0.29 | ±0.98 | ±1.95 |
| S2 | ±0.03* | ±0.06 | ±0.12 | ±0.19 | ±0.62 | ±1.24 |
| S3 | ±0.03* | ±0.05 | ±0.06 | ±0.09 | ±0.29 | ±0.59 |
| S4 | ±0.03* | ±0.15 | ±0.30 | ±0.45 | ±1.51 | ±3.02 |

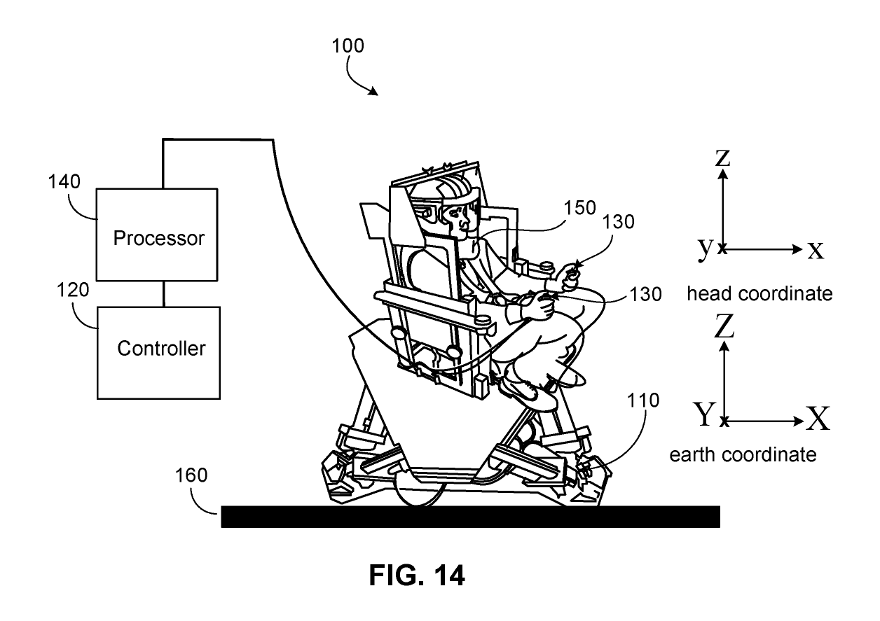

The asterisks in the entries of Table 1 denote that the smallest stimulus magnitude was set in accordance with the minimum motion that could be reliable provided by the MOOG platform (described below with reference to FIG. 14) used in the experiments. Predicted confidence judgment distributions for two separate CSD models—CSD2 (denoted by the curve connecting the +markers) and CSD3 (denoted by the curve connecting the X markers) using fitted parameters for each subject are overlapped for comparison. For each of the CSD models a standard Gaussian psychometric function, \hat{Ψ}(x)=ϕ(x:\hat{μ},\hat{σ}), and a Gaussian confidence function, \hat{χ}(x)=ϕ(x,\hat{μ},\hat{k},\hat{σ}), were fitted to the data using maximum likelihood methods. To investigate the impact of the confidence-scaling factor \hat{k}, the data was fit in two different ways. For one fit, the confidence-scaling factor was fixed as \hat{k}=1). This is referred to as the CSD2 model. For the CSD2 model, the confidence function was defined to have the exact same parameters as the psychometric function, under the assumption of perfect calibration. For the second fit, the confidence-scaling factor provided a third parameter. This fit is referred to as the CSD3 model. The CSD3 model allows different width parameters for the psychometric function, and the confidence function to represent over-confidence (\hat{k}<1) or under-confidence (\hat{k}>1).

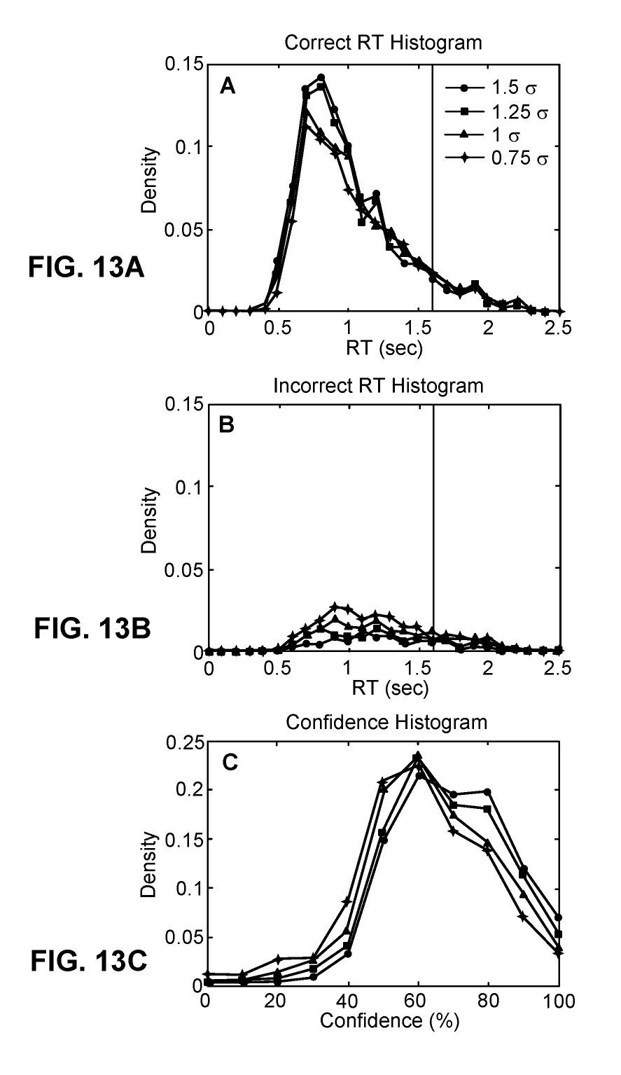

FIGS. 13A-13C show results of a confidence probability judgment test associated with a fixed-duration direction-recognition task. Specifically, a subjective visual vertical (SVV) task (with the subject seated in an upright position in the dark) was used to assay visual-vestibular integration. For the SVV studies, Gabor patch characteristics (as described in the publication: Baccini M, et. al, The assessment of subjective visual vertical: comparison of two psychophysical paradigms and age-related performance. Atten. Percept Psychophys. 2013.) was used because these characteristics provided acceptable data for the confidence goals.

Twelve subjects completed the SVV study. Stimuli (repeated sixty times at each amplitude) provided at 0.75, 1, 1.25, and 1.5 times (as shown via the figure legend) the baseline threshold were randomly intermixed. Histograms for correct response time (RT) (FIG. 13A), incorrect RT (FIG. 13B), and confidence (FIG. 13C) were generated. The subjects were instructed to press a button as soon as a decision was reached about the perceived orientation of the Gabor patch. RT was marked when a button was pressed. The Gabor patch disappeared when subjects pressed the button to minimize sensory evidence for subsequent confidence reporting. Subjects verbally reported confidence with 5% resolution. The RT histograms for correct SVV responses were found to be similar for different stimulus magnitudes (FIG. 13A), as were the confidence histograms (FIG. 13C). This indicated that the subjects maintained consistent decision criteria (i.e., similar boundaries) across large SVV stimulus variations.

### Vestibular Testing Systems

FIG. 14 shows an example of a vestibular testing system 100 that can be used to implement some or all of the methods and processes described herein. The vestibular testing system 100 includes a motion platform 110 (e.g., a MOOG series 6DOF2000e), a controller 120 for controlling the motion of the motion platform 110, and an input device 130 for receiving input from a subject 150 whose vestibular system is to be tested. The processor 140 can receive input information from the input device 130 and may provide instructions to the controller 120 for moving the motion platform 110. During operation, the motion platform 110 supports the subject 150 and the controller 120 can provide a stimulus signal to the motion platform 110 for movement. In some implementations, the processor 140 can be integrated with the input device 130.

Generally, each motion of the motion platform 110 can be described by a motion profile that includes information about the direction of motion and other features related to the motion. For example, a motion can be a translational motion along any of the three perpendicular axes x, y, and z of a coordinate system centered on head of the subject 150. Referring to FIG. 14, the x axis is pointing forward from the head, the y axis is pointing left from the head (into the drawing plane), and the z axis is pointing upward from the head. Such coordinate system respect to the head is referred as the “head coordinate” in this specification.

The motion profile can include amplitude and frequency of the velocity and acceleration of the motion. The amplitude of the acceleration and velocity vary with time, whereas the frequency remains constant. For example, a translational motion starts with a zero velocity, accelerates to a maximum velocity, and decelerates to zero again. For example, the acceleration is sinusoidal and can be expressed as a(t)=A sin(2πft), where a(t) is the acceleration at time t, A is the acceleration amplitude, and/is the frequency. With such acceleration, starting from zero, the translational velocity v(t) at time t is v(t)=A/2πft[1−cos(2πft)]

Similarly, a rotational motion can include a sinusoidal angular acceleration and an angular velocity, both of which are expressed in a manner similar to the translational acceleration and velocity of the above-noted equations for a(t) and v(t).

The motion platform 110 moves the subject along a trajectory in a spatial coordinate system while following a velocity profile. The velocity profile relates the magnitude of velocity to time. At the beginning and end of the motion, the magnitude of the velocity is zero. At some point in between, the velocity reaches a maximum magnitude, referred to herein as “peak velocity” or “peak stimulus velocity.” In many applications, the velocity profile is one cycle of such a velocity oscillation. The reciprocal of the period of this sine wave is referred to herein as “frequency” or “motion frequency.” As noted above, the shape of the velocity profile can be sinusoidal. However, other shapes are possible, such as those defined by superpositions of weighted and/or timeshifted components.

The motion platform 110 can have a translational motion in either x, y, or z direction. Accordingly, the translation motion in either direction is referred as “x-translation”, “y-translation”, or “z-translation”, respectively. In addition, the motion platform can have various rotational motions. Rotation about the x axis is referred as “roll” rotation, rotation about the y axis is referred as “pitch” rotation, and rotation about the z axis is referred as “yaw” rotation. The movements can be caused by the stimulus signal provided by the controller 120.

In some implementations, the controller 120 can change the orientation of the motion platform 110. Alternatively, a person can manually change the orientation. For example, the motion platform can be rotated 90 degrees to the side such that the subject 150 is lying on his or her side. Considering the variety of orientations of the motion platform 110, it is useful to refer a motion of the motion platform 110 (or the subject 150) using X, Y, and Z coordinates with respect to the fixed earth 160 (or ground.) Such coordinates are referred as “earth coordinates” in this specification. The Z direction is referred as “earth-vertical” and either the X or Y direction is referred as “earth-horizontal”.

In the example illustrated in FIG. 14, the X axis refers to a direction parallel to the ground, and the Y axis refers to another direction parallel to the ground, but perpendicular to the X axis. The Z axis points vertical to the ground. In this example, the head coordinates x, y, and z axes coincide with the earth coordinates X, Y, and Z axes. The illustrated body orientation of subject 150 is referred as the “upright position”.

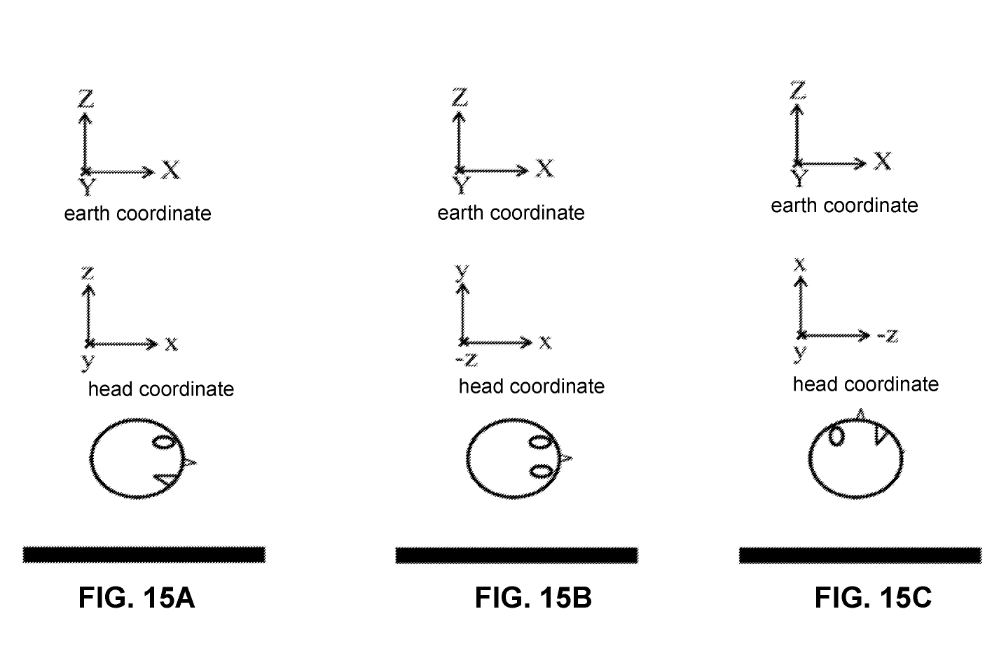

In some implementations, the motion platform 110 can be moved to be oriented such that the body orientation of the subject 150 is different from the upright position. FIGS. 15A-15C show a schematic of three different body orientations. FIG. 15A shows the up-right position previously described. FIG. 15B shows a “side-up position” where the motion platform 110 is rotated by 90 degrees such that the right side of the head is pointing towards the ground. In this orientation, the z axis may coincide with the −Y axis and the y axis may coincide with the Z axis. Alternatively, the left side of the head may point towards the ground. FIG. 15C shows a “back-down position” where the back of the head is pointing towards the ground. In this orientation, the x axis may coincide with the Z axis and the −z axis may coincide with the X axis. A “front-down position” refers when the front of the head is pointing towards the ground.

Accordingly, the motion platform 110 may move the subject 150 in a variety of configurations depending on the body orientation, type, or direction of motion in head coordinates. In some implementations, the motion platform 110 can be configured to provide only one or several types of motions and body orientations. In this specification, a motion along, or aligned with, a specific direction may refer to motion in positive and negative directions of the specific direction. Similarly, a motion parallel to a specific direction may refer to motion which is parallel or antiparallel to the specific direction.

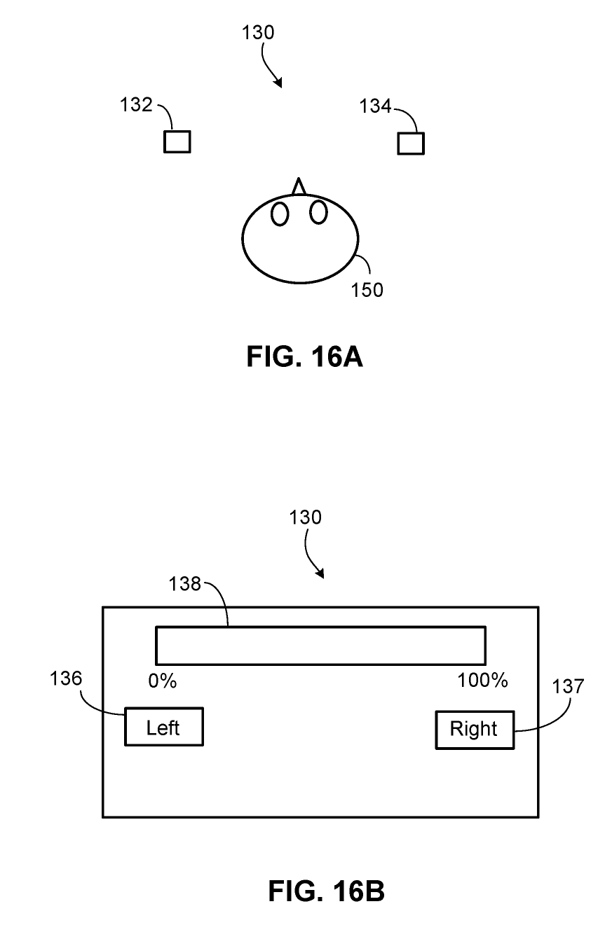

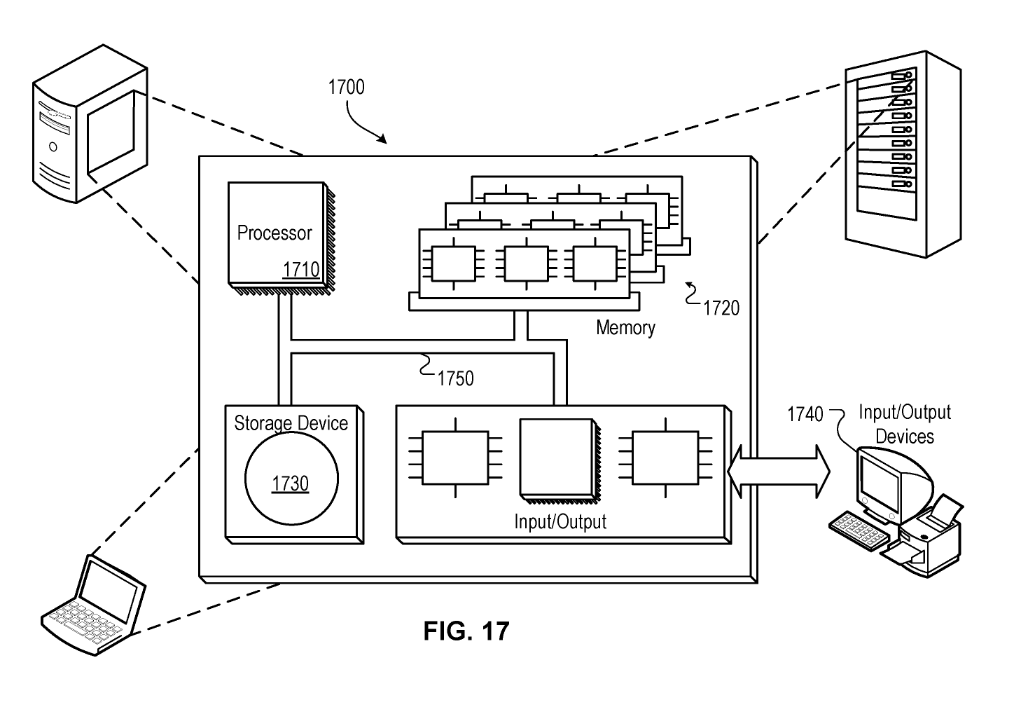

During operation, the subject 150 provides an input to the input device 130 to communicate his or her perception of motion to the processor 140. FIG. 16A shows an example of an input device 130, which includes a pair of buttons 132 and 134. Other examples of input device 130 include a joystick, pair of joysticks, a keyboard, a pair of switches, or foot pedals. After a motion of the motion platform 110, the subject 150 can press one of the buttons 132 and 134 to indicate his or her perception. For example, a particular button pressed can indicate the subject's perception of the motion's direction. In some examples, the subject 150 can press button 132 upon perceiving an upward translational motion and press button 134 when perceiving a downward translational motion.

FIG. 16B shows another example of an input device 130, which can be a touch screen such as a tablet device or a keyboard, e.g., a numeric keypad. The subject can indicate his or her perception by pressing either location 136 or 137 on the input device 130. For example, after a y-translation motion, the subject 150 can select location 136 if he or she perceives motion to his or her left. Alternatively, the subject 150 can select location 137 if he or she perceives motion to his or her right. As another example, after a z-translation motion, the locations 136 and 137 can be indicative of “up” or “down,” respectively. In some implementations, the input device 130 can simultaneously display more than two locations indicative of several types of motion (e.g., “left”, “right”, “up”, “down”, “translation”, “rotation”, etc.) In some implementations, the subject 150 can input his or her perception of a motion by swiping the display of the input device 130. For example, the subject 150 can swipe his or her fingers on the display to the left to indicate that the perceived motion is to his or her left direction.

In the example shown in FIG. 16B, the input device 130 includes a confidence rating menu 138. The subject 150 can indicate his or her confidence rating of the perceived motion using the confidence rating menu 138. In this example, the confidence rating menu is a quasi-continuous rating menu where 0% to 100% indicates the level of confidence in 1% increments. A quasi-continuous rating between 50% (guessing) and 100% (certain) is another example. Other ranges can be used. As described below, various types of confidence ratings other than the quasi-continuous rating can be used. In some implementations, the confidence rating menu 138 can be designed according to the type of confidence rating to be used.

In some implementations, the input device 130 can receive a binary response from the subject 150 through locations 136 and 137. After receiving the binary response, the input device 130 can further receive a confidence rating through the confidence rating menu 138. For example, the subject 150 can augment his or her binary response by providing a confidence rating including: (1) a quasi-continuous rating (e.g., 50% confidence to 100% confidence); (2) a binary rating (e.g., guessing versus certain); (3) a quinary rating (e.g., 1 to 5 where 1 is “guessing” and 5 is “certain,” or vice versa) or an N-level discrete rating (e.g., 1 to N where 1 is “guessing” and N is “certain” or vice versa); or (4) a wagering rating (e.g., the user wagers 1-10 points with each response and loses the wagered number of points if the response is incorrect or gains the wagered number of points if the response is correct). The confidence rating can also be a combination of the forms (1)-(4). As described elsewhere herein, the received confidence rating can be used to: (1) improve the quality of estimating the psychometric function; (2) improve the efficiency of targeting stimulus levels in real-time via a closed-loop system during psychometric test; (3) reduce the negative impacts of indecision; (4) help evaluate subject's with psychometric (e.g., vestibular) dysfunctions; or (5) help evaluate malingerers. It is also understood that the confidence rating can be received before or simultaneous with the binary response.

As described above, the input device 130 can receive both the binary response and the confidence rating for a given motion, in other words, for each trial. The received data (e.g., binary response, confidence rating) can be communicated to the processor 140. The processor 140 can estimate a psychometric function and its threshold based on the communicated data. The communication can be done in a wired or wireless (e.g., WiFi, Bluetooth, or Near Field Communication) manner.

The controller 120 can instruct (e.g., by providing stimuli signals) a predefined set of motions to the motion platform. Alternatively, the controller 120 can instruct the motion platform based on the input received by the input device 130. For example, the processor 140 is configured to instruct the controller 120 to cause execution of those motions for which expected information about a subject's perception of those motions would most contribute to improving an estimate of a subject's vestibular threshold. Such an estimate can be used to construct a vestibulogram, which shows the subject's vestibular threshold at different frequencies.

Referring back to FIG. 14, the controller 120 instructs the motion platform 110 to execute motions. For example, the motions can be selected for those motions for which expected information about the subject's perception of those motions would most contribute to improving an estimate of a subject's vestibular threshold.

### Methods

### Confidence Maximum Likelihood Fit Analysis

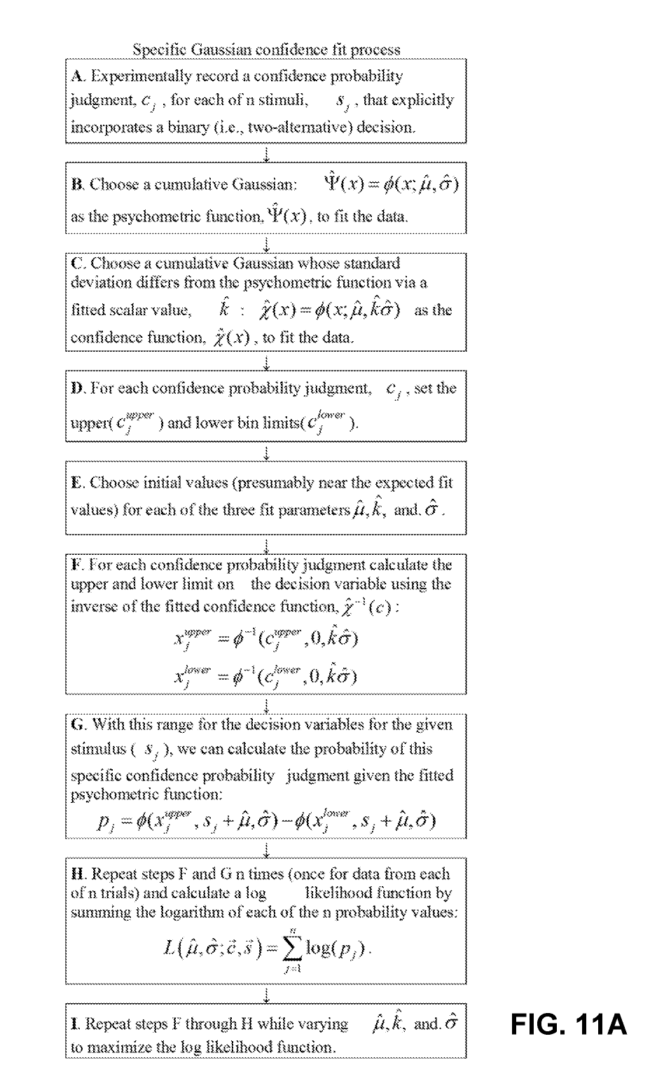

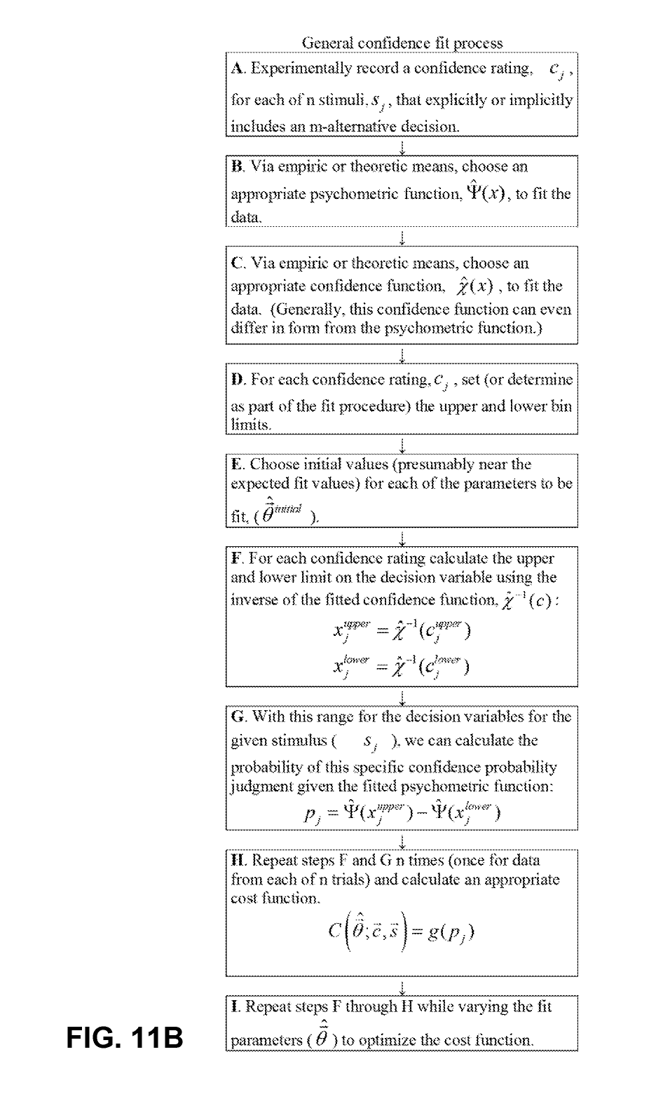

This section presents a maximum likelihood analysis developed to help estimate psychometric function fit parameters. This technique simultaneously fits both a psychometric function (\hat{ψ}(x)) and a confidence function (\hat{χ}(x)) to confidence probability judgments. FIGS. 11A and 11B presents flow charts that outline this fitting technique. The specific model we use is presented via the flow chart in FIG. 11A and a generalized flow chart is provided in FIG. 11B. In some implementations, the processes and operations shown in the flow charts of FIGS. 11A and B can be implemented at least in part by one or more processors. In some cases, the processes and operations are implemented at least in part by a human operator.

For the process depicted in the generalized flow chart of FIG. 11B, at step A, an operator experimentally records a confidence rating, cⱼ, for each of n stimuli, sⱼ, that explicitly or implicitly includes an m-alternative decision. At step B, the operator, via empiric or theoretic means, chooses an appropriate psychometric function, \hat{Ψ}(x), to fit the data. At step C, the operator, via empiric or theoretic means, chooses an appropriate confidence function, \hat{χ}(x), to fit the data. The confidence function can differ in form from the psychometric function. At step D, the operator, for each confidence rating, cⱼ, sets or determines as part of the fit procedure the upper and lower bin limits. At step E, the operator chooses initial values, for example, near the expected fit values, for each of the parameters to be fit, (\hat{\vec{θ}}^{initial}). At step F, the operator for each confidence rating calculates the upper and lower limit on the decision variable using the inverse of the fitted confidence function, \hat{χ}⁻¹(c):

xⱼ^{upper}=\hat{χ}⁻¹(cⱼ^{upper})

xⱼ^{lower}=\hat{χ}⁻¹(cⱼ^{lower})

At step G, the operator, with this range for the decision variables for the given stimulus (sⱼ), calculates the probability of this specific confidence probability judgment given the fitted psychometric function:

pⱼ=\hat{Ψ}(xⱼ^{upper})−\hat{Ψ}(xⱼ^{lower})

At step H, the operator repeats steps F and G n times, for example, once for data from each of n trials and computes an appropriate cost function, C(\hat{\vec{θ}};\vec{c},\vec{s})=g(pⱼ). At step I, the operator repeats steps F through H while varying the fit parameters (\hat{\vec{θ}}) to optimize the cost function.

For the process depicted in the flow chart of the specific model of FIG. 11A, at step A, the operator experimentally records a confidence probability judgment, cⱼ, for each of n stimuli, sⱼ, that explicitly incorporates a binary (i.e., two-alternative) decision. At step B, the operator chooses a cumulative Gaussian: \hat{Ψ}(x)=ϕ(x;\hat{μ},\hat{σ}) as the psychometric function, \hat{Ψ}(x), to fit the data. At step C, the operator chooses a cumulative Gaussian whose standard deviation differs from the psychometric function via a fitted scalar value, \hat{k}: \hat{χ}(x)=ϕ(x;\hat{μ},\hat{k}\hat{σ}) as the confidence function, \hat{χ}(x), to fit the data. At step D, the operator, for each confidence probability judgment, cⱼ, sets the upper(cⱼ^{upper}) and lower bin limits(cⱼ^{lower}). At step E, the operator chooses initial values (presumably near the expected fit values) for each of the three fit parameters \hat{μ},\hat{k}, and. \hat{σ}. At step F, for each confidence probability judgment, the operator calculates the upper and lower limit on the decision variable using the inverse of the fitted confidence function, \hat{χ}⁻¹(c):

xⱼ^{upper}=ϕ⁻¹(cⱼ^{upper},0,\hat{k}\hat{σ})

xⱼ^{lower}=ϕ⁻¹(cⱼ^{lower},0,\hat{k}\hat{σ})

At step G, the operator, with this range for the decision variables for the given stimulus (sⱼ), calculates the probability of this specific confidence probability judgment given the fitted psychometric function, pⱼ=ϕ(xⱼ^{upper},sⱼ+\hat{μ},\hat{σ})−ϕ(xⱼ^{lower},sⱼ+\hat{μ},\hat{σ}). At step H, the operator repeats steps F and G n times, for example, once for data from each of n trials, and calculates a log likelihood function by summing the logarithm of each of the n probability values:

$$L ( \hat{μ} , \hat{σ} ; \vec{c} , \vec{s} ) = {\sum }_{j = 1}^{n} log ( {p}_{j} ) .$$

At step I, the operator repeats steps F through H while varying \hat{μ},\hat{k}, and. \hat{σ} to maximize the log likelihood function.

To describe the confidence-based technique we assume that the fitted psychometric function can be represented by a Gaussian cumulative distribution function (ϕ) having two fit parameters (\hat{μ}, \hat{σ}):

\hat{ψ}(x)=ϕ(x;\hat{μ},\hat{σ}) (1)

where \hat{μ} represents shifts in the psychometric function (i.e., mean value of the noise distribution) and represents the width of the psychometric function (i.e., standard deviation of the noise distribution), which is often referred to as the threshold for direction-recognition tasks. Assuming that subjects based their confidence assessment on the signal used to make their decision, we modeled the fitted confidence function as a Gaussian cumulative distribution function having one additional free parameter, a confidence-scaling factor (\hat{k}) that scales this average confidence function to account for under-confidence or over-confidence, as previously demonstrated in FIG. 1D:

\hat{χ}(x)=ϕ(x,\hat{μ},\hat{k}\hat{σ}) (2)

We assume a Gaussian confidence function for simplicity, but other shapes of the confidence function were investigated via simulations to evaluate the impact of this assumption. Noise was not explicitly included in this relationship; noise may be present in the mapping from a decision variable to the confidence response, and we evaluate the impact of additive noise via simulations. FIGS. 2A and 2B schematically illustrate the neural processing underlying the model.

FIGS. 3A to 3C schematically demonstrate the maximum likelihood calculation for an individual trial. For each confidence probability judgment (c₁) provided by the subject, we can calculate the corresponding decision variable via the inverse Gaussian CDF:

\hat{x}ⱼ=\hat{χ}⁻¹(cⱼ)=ϕ⁻¹(cⱼ;\hat{μ},\hat{k}\hat{σ}) (3)

where cⱼ represents a confidence probability judgment, \hat{χ}⁻¹(cⱼ) represents the inverse fitted confidence function, and ϕ⁻¹ represents the inverse cumulative Gaussian. The precise probabilistic interpretation of a confidence probability judgment depends on the resolution of the subjective scale provided the subject. Our subjects provided a confidence probability judgment using a scale that had a resolution of 1%. Therefore, when a subject provided a confidence probability judgment of 70%, we set the lower (cⱼ^{lower}) and upper (cⱼ^{upper}) bin limits to 69.5 and 70.5%, respectively. Using equation 3, lower (xⱼ^{lower}) and upper (xⱼ^{upper}) decision variable limits can be calculated for a given confidence probability judgment, cⱼ.

As illustrated schematically (FIG. 2B), we can then calculate the probability (pⱼ), which in this context is commonly called the “likelihood,” that a decision variable for an individual trial falls in this range using the relationship:

pⱼ=\hat{Ψ}(xⱼ^{upper})−\hat{Ψ}(xⱼ^{lower})=ϕ(xⱼ^{upper};sⱼ+\hat{μ},\hat{σ})−ϕ(xⱼ^{lower};sⱼ+\hat{μ},\hat{σ}) (4)

where sⱼ is the stimulus provided on that trial.

Repeating this process for each of the N trials, we can then calculate the log likelihood by simply summing the log of each of the individual trial likelihoods, which can be written as:

$$L ( \hat{μ} , \hat{σ} , \hat{k} ; \vec{c} , \vec{s} ) = {\sum }_{j = 1}^{n} log ( {p}_{j} ) \qquad ( 5 )$$

We find the maximum likelihood fit by numerically finding the three fit parameters (\hat{μ},\hat{σ},\hat{k}) that maximize the value of this log likelihood function. This method assumes that the confidence judgment utilizes a similar decision variable as the binary decision-making process. The methods described herein also assume that all processes and mechanisms (e.g., decision boundary, confidence estimation, etc.) are stationary (i.e., constant) across time. This stationarity assumption is included in some psychometric function fits as well. FIGS. 3A to 3C show example fitted functions.

### Data Analysis

To provide a direct comparison of the confidence-based fitting method to standard binary forced-choice fitting methods, we fit psychometric curves to the binary data using a maximum likelihood approach. For our forced-choice direction-recognition task, the subject's directional responses are binary (e.g., left or right) and the psychometric function ranges from 0 to 1. A Gaussian distribution was fitted to the data using MATLAB® programming language to generate a generalized linear model using a probit link function. An example of a psychometric function fit to binary data is shown in FIG. 3B.

The general technique used to fit a psychometric function and a confidence function to the confidence data was described with respect to FIGS. 2A and 2B. To find the maximum likelihood parameter estimates we minimized the negative of the likelihood via a numeric optimization algorithm (fmincon function in MATLAB® programming language). The initial value for the confidence-scaling factor (\hat{k}) was assumed equal to 1.0; initial values for the psychometric function parameters (\hat{μ},\hat{σ}) were set equal to the values obtained by the GLM fit of the binary forced-choice data.

### Human Studies

Each subject was seated in a racing-style chair with a five-point harness; his/her head was fixed relative to the chair and platform via an adjustable helmet. Each subject wore a pair of noise cancelling earpieces that also provided the ability to communicate with the experimenter. All motions were performed in darkness. Subjects performed a binary forced-choice direction-recognition task in response to upright whole-body yaw rotation. Aural white noise began playing in the subject's earpiece 300 ms before motion commenced and ended when the motion ended. This aural cue was provided to mask any potential directional auditory cues and also informed the subject when a trial began and ended. When the motion and white noise ended, a tablet computing device illuminated and subjects were required to report the motion direction perceived and a confidence probability judgment. Single cycles of sinusoidal acceleration at 1 Hz were used as the motion stimuli. Motion stimuli were generated using MOOG® 6 degrees of freedom motion platform. There was a pause of at least 3 s between motions. An adaptive sampling procedure—a standard 3-Down/1-Up (3D/1U) staircase using PEST rules was utilized. The initial stimulus amplitude was 4°/s. FIG. 3A shows an example stimulus track for the first twenty trials. There were a hundred trials in each experiment.

Subject responses, both the direction responses (i.e., left or right) and quantitative confidence probability judgments having a resolution of 1%, were recorded using a tablet computing device (e.g., an iPad® tablet computing device). Before each trial, the tablet computing device backlighting was turned off. When the trial ended, the tablet computing device was automatically illuminated to display sliders (one on the left and one on the right) that ranged from 50% to 100%. The subject tapped on the left side of the tablet computing device to report perceived motion to the left and tapped on the right side to report perceived motion to the right. Subjects could then move the selected slider up/down to indicate their confidence. To avoid biasing the subject's confidence responses, a slider position was not displayed until the subject touched the screen to indicate their confidence (i.e., no initial slider position was provided to the subject). The subject's responses including both directions (left/right) and confidence (50% to 100%) were displayed on the screen. The subject could adjust their response until satisfied. The subject then invoked a button on the tablet computing device labeled “Confirm”. At the beginning of the testing, the subjects practiced for a few trials to improve their understanding of the task.

These human studies utilized a half-range task in which subjects used a scale between 50% and 100%, inclusive. Confidence probability judgment tasks can be full-range tasks or half-range tasks. Full-range scales range between 0% and 100%, while half-range scales range between 50% and 100%. For our half-range task, a subject could report that they perceived negative motion and report 84% confidence. For a full range task with the subject asked to report their confidence that the motion was positive, the equivalent response would be a 16% confidence that the motion was positive. To plot, model, and fit the data, we used this mathematical equivalence to convert each half-range confidence rating to a full-range rating.

Subject instructions indicated that the motion direction would be selected randomly and that the directions of previous motions would not impact the next motion direction. Instructions also indicated that expectations regarding the distribution of confidence assessments and that they report the confidence that they experienced for each specific trial. Subjects were informed that “ . . . if you are guessing much of the time, this is OK, and if you are very certain much of the time this is OK, too.” Subjects were not provided information regarding their confidence indications. During the initial training that did not exceed 10 practice trials, subjects were informed whether their left/right responses were correct or incorrect. During test sessions, subjects were not informed whether their responses were correct or incorrect.

Four healthy human subjects (2 male, 2 female, 26-34 years old) were each tested on six different days. Informed consent was obtained from all subjects prior to participation in the study. The study was approved by the local ethics committee and was performed in accordance with the ethical standards laid down in the 1964 Declaration of Helsinki.

For one subject, since the computer randomized the motion direction for each trial just before the trial and since the adaptive staircase targets stimuli where an average subject may get about 20% of the trials incorrect, this subject did not have information to guide his binary reports or confidence judgments on each individual trial. As noted in the results, this subject's responses did not differ from the other subjects in any noticeable manner.

### Simulations

All simulations were performed using MATLAB® computing language, Release R2015a (The Mathworks®, Inc.) using parallel IBM® BladeCenter® HS21 XMs with 3.16 GHz Xeon® processors and 8 GB of RAM. These simulations used the same standard adaptive sampling procedure used for the human studies. Specifically, we used a 3-Down/1-Up (3D/1U) staircase having a hundred trials. The simulated 3D/1U staircases began at a stimulus level of four. The size of the change in stimulus magnitude was determined using PEST (parameter estimation by sequential testing) rules.

For all four simulated data sets included and described herein, the psychometric function, Ψ(x)=ϕ(x;μ=0.5,σ=1), and the fitted psychometric function, \hat{Ψ}(x)=ϕ(x;\hat{μ},\hat{σ}), were modeled as cumulative Gaussians.

For the first simulated data set (as represented, for example, in the first column of FIG. 8), the confidence function was modeled as “well-calibrated” meaning that the confidence function equaled the psychometric function, χ(x)=Ψ(x)=ϕ(x;μ=0.5,σ=1). For the second simulated data set (as represented, for example, in the second column of FIG. 8), the confidence function was modeled as “under-confident” with a confidence-scaling factor (k) of 2, yielding a confidence function of χ(x)=ϕ(x;μ=0.5,σ=2). For the last two simulated data sets (as represented, for example in the third and fourth columns of FIG. 8), the confidence function was linear crossing the 0.5 confidence level with a bias of 0.5 (μ=0.5) and with a slope of 0.1443 (m=0.1443), χ(x)=m(x−μ)+0.5=0.1443x+0.428, which yielded confidence saturations at −2.96 (“zero”) and +3.96 (“one”).

For the first three simulated data sets (FIGS. 7A-7C, FIGS. 7D-7F, and FIGS. 7G-7I, and the first three columns of FIG. 8 corresponding to panels A and E, B and F, and C and G, respectively), we fit the data by modeling the fitted confidence function as the 3-parameter Gaussian CDF of Equation 3, \hat{χ}(x)=ϕ(x,\hat{μ},\hat{k}\hat{σ}). For the last simulated data set (see FIGS. 7J-7L and the fourth column of FIG. 8 corresponding to Panels D and H, respectively), we fit the data by modeling the fitted confidence function as a linear model of confidence, \hat{χ}(x)=\hat{m}(x−\hat{μ})+0.5, which matched the form of the simulated confidence function.

Two simulation data sets included additive noise. For the simulations shown in FIGS. 7G-7I and the third column of FIG. 8, the noise distribution was modeled as zero-mean uniform noise having width of 20% (−10% to +10%), U(−0.1, +0.1). We chose a large noise range to demonstrate the small impact of such confidence noise. The relative stability of human confidence ratings suggest that this simulated noise level overestimates the actual contributions of confidence noise. This noise distribution indicates that, if the confidence function yielded a confidence rating of 70%, the noise would lead to a report of between 60% and 80%. Such a report yields approximately 3 functional confidence bins between 50% (just guessing) and 100% (certain). We chose uniform noise because we could control the noise range without impacting the noise distribution and so as to maintain the confidence in the range 0% to 100%. To keep the noise zero-mean when the confidence function yielded a confidence of greater than 90% (or less than 10%), the limits on the noise distribution were reduced to keep the confidence judgment in the range of 0 to 100%. For example, if the simulation yielded a mean confidence of 94% for an individual trial, the noise distribution was modeled as zero-mean uniform with a range of −6% to 6% (i.e., U(−0.06, +0.06)) for that trial. The distribution was not modeled as such for most trials in which confidence was lower than 90%. For the simulations shown in FIGS. 7J-7L, and the fourth column of FIG. 8, the noise distribution was again modeled as zero-mean uniform noise but with a width of 10% (−5% to +5%), U(−0.05,+0.05).

### Results

### Human Studies

Fitted psychometric function (\hat{μ},\hat{σ}) and confidence scaling (\hat{k}) parameters for each of our four subjects for yaw rotation about an earth-vertical rotation axis are shown in FIGS. 4A to 4L depicting the mean and 5 depicting the standard deviation. Parameter fits are plotted versus the number of trials in increments of 5 trials starting at the 15^{th} trial. To demonstrate raw performance for individual test sessions, FIG. 9 presents the parameter fits for each of the six individual tests for each subject. As described in the “Methods” section, all parameter estimates are determined using maximum likelihood methods.

Consistent with studies utilizing adaptive procedures, the confidence-agnostic estimates of the width of the psychometric function (\hat{σ}) took between fifty and a hundred trials to stabilize (FIGS. 4A, 4D, 4G, and 4J, curves 400a, 400b, 400c). More specifically, using these psychometric methods, the estimated width parameter (\hat{σ}) was significantly lower after twenty trials than after a hundred trials (repeated measures ANOVA, N=4 subjects, p=0.011).

In contrast, estimates of the width parameter (\hat{σ}) using the confidence fit technique could require fewer than twenty trials to reach stable levels (FIGS. 4A, 4D, 4G, and 4J, curves 402a, 402b, 402c). Specifically, the width parameter (\hat{σ}) estimated using confidence probability judgments was not significantly different after twenty trials than for a hundred trials (repeated measures ANOVA, N=4 subjects, p=0.251). Furthermore, the estimated width parameter after twenty trials using confidence probability judgments was not significantly different from the estimated width parameter after a hundred trials using psychometric fit methods absent confidence analysis (repeated measures ANOVA, N=4 subjects, p=0.907).

Furthermore, the parameter estimates obtained using confidence-agnostic psychometric fits (FIG. 5, black traces) were more variable than the fits obtained using the CSD model (FIG. 5, gray traces). The precision of the psychometric width estimate using the confidence model after twenty trials (average standard deviation of 0.124 across subjects) was similar to those of the psychometric fit estimates without consideration of confidence after a hundred trials (0.129).

The estimates of the shift of the psychometric functions (\hat{μ}) showed a qualitatively similar pattern; the estimates that utilized confidence reached stable levels a sooner and were more precise than the estimates provided by the analysis that does not account for confidence. We also note that, for three of our subjects (FIGS. 4B, 4E, and 4H), fitted confidence-scaling factors were near 1. The other subject had a fitted confidence-scaling factor near 2 (FIG. 4H), suggesting under-confidence.

### Simulations

We also simulated tens of thousands of test sessions to test the confidence fit procedures described herein. The simulations simulated the human studies with a difference being that we defined the simulated psychometric (Ψ(x)) and confidence (χ(x)) functions. Since we knew these simulated functions, this allowed us to quantify parameter fit accuracy. For all simulated data sets, we fit the binary forced-choice data without confidence analysis and compared and contrasted these fits with the CSD fits Histograms show fitted parameters after twenty (FIGS. 6A, 6C, and 6E) and a hundred (FIGS. 6B, 6D, and 6F) trials for 10,000 simulations. After as few as twenty trials, the CSD fit parameters demonstrated relatively tight distributions (FIGS. 6C and 6E) in comparison to the binary fits that show ragged distributions (FIG. 6A). After a hundred trials, the binary fit parameters demonstrated relatively tight distributions (FIG. 6B) that were similar to the CSD fit parameters determined after twenty trials (FIGS. 6C and 6E). The CSD fit parameters after a hundred trials (FIGS. 6D and 6F) demonstrated higher precision (i.e., lower variance) than the binary fit parameters after a hundred trials (FIG. 6B). FIGS. 10A-10D presents similar histograms for a hundred trials for the other two simulation data sets.

Mimicking the format previously used for the human data (FIGS. 4A to 4L and 5); simulation parameter fits are plotted versus the number of trials in increments of 5 trials starting at the 15^{th} trial. Curves 700a, 700b, and 700c of FIGS. 7A-7L and the black curves of FIG. 8 show the fitted psychometric function parameters for the binary forced choice data. Curves 700a, 700b, 700c and curves 702a, 702b, 702c show the fitted psychometric parameters and confidence function parameters, respectively, fit using the CSD model. To provide direct quantitative comparisons, Tables 2-5 summarize data from all simulations in tabular form.

Tables 2-5 summarize results from 10,000 simulated test sessions for direct quantitative comparisons. These fit parameters are the same as shown graphically in FIGS. 7A-7L and 8. The last row of Tables 2 and 4 quantitatively presents the fitted psychometric parameters using the confidence-agnostic binary methods; these were graphically presented in FIGS. 7A-7L and 8 as black curves. The 1^{st} to the 4^{th} rows of Tables 2-4 quantitatively present the fitted psychometric and confidence scaling parameters found using the CSD fit; these were graphically presented in FIGS. 7A-7L and 8. Specifically, the 1st row of Tables 2-4 quantitatively presents fit parameters for a well-calibrated subject (k=1) when both confidence and confidence fit functions were cumulative Gaussians (FIGS. 7A-7C and the first column of FIG. 8). The 2^{nd} row of Tables 2-4 quantitatively presents fit parameters for an under-confident subject (k=2) when both confidence and confidence fit functions were cumulative Gaussians (FIGS. 7D-7F and the second column of FIG. 8). The 3^{rd} row of Tables 2-4 quantitatively presents fit parameters an under-confident subject when the confidence function is linear, χ(x)=m(x−μ)+0.5=0.1443x+0.428, with added zero-mean uniform noise (U(−0.1, +0.1)), and the confidence fit function was a cumulative Gaussian (FIGS. 7G-7I, and the 3rd column of FIG. 8). The 4th row of Tables 2-4 present fit parameters for an under-confident subject with the same linear confidence function with added zero-mean uniform noise (U(−0.05, +0.05)) when the confidence fit function was linear, \hat{χ}(x)=\hat{m}(x−\hat{μ})+0.5 (FIGS. 7J-7L and the fourth column of FIG. 8). Table 5 shows parameters that summarize different characteristics of the fit, including goodness of fit.

TABLE 1
Fitted width parameter (\hat{σ}).

| Trials | 20 | | 40 | | 60 | | 80 | | 100 | |
|---|---|---|---|---|---|---|---|---|---|---|
| FIGS. 7A-7C | 0.926 | (0.213) | 0.960 | (0.163) | 0.973 | (0.139) | 0.977 | (0.121) | 0.983 | (0.111) |
| FIGS. 7D-7F | 0.946 | (0.188) | 0.971 | (0.144) | 0.980 | (0.125) | 0.984 | (0.111) | 0.987 | (0.102) |
| FIGS. 7G-7I | 1.053 | (0.279) | 1.041 | (0.218) | 1.045 | (0.189) | 1.050 | (0.166) | 1.058 | (0.153) |
| FIGS. 7J-7K | 0.955 | (0.203) | 0.984 | (0.156) | 0.995 | (0.134) | 0.999 | (0.119) | 1.003 | (0.109) |
| Binary | 0.588 | (0.484) | 0.841 | (0.340) | 0.914 | (0.257) | 0.944 | (0.209) | 0.959 | (0.179) |

TABLE 2
Fitted confidence scaling factor (\hat{k})

| Trials | 20 | | 40 | | 60 | | 80 | | 100 | |
|---|---|---|---|---|---|---|---|---|---|---|
| FIGS. 7A-7C | 1.102 | (0.234) | 1.045 | (0.137) | 1.029 | (0.107) | 1.021 | (0.090) | 1.016 | (0.079) |
| FIGS. 7D-7F | 2.189 | (0.389) | 2.089 | (0.245) | 2.058 | (0.194) | 2.043 | (0.164) | 2.034 | (0.146) |
| FIGS. 7G-7I | 1.752 | (0.464) | 1.814 | (0.360) | 1.845 | (0.308) | 1.859 | (0.272) | 1.868 | (0.248) |
| FIGS. 7J-7K | 0.148 | (0.022) | 0.148 | (0.018) | 0.147 | (0.015) | 0.147 | (0.014) | 0.146 | (0.013) |

TABLE 3
Fitted bias parameter (\hat{μ})

| Trials | 20 | | 40 | | 60 | | 80 | | 100 | |
|---|---|---|---|---|---|---|---|---|---|---|
| FIGS. 7A-7C | 0.499 | (0.255) | 0.494 | (0.175) | 0.496 | (0.143) | 0.497 | (0.123) | 0.497 | (0.111) |
| FIGS. 7D-7F | 0.505 | (0.237) | 0.500 | (0.167) | 0.500 | (0.137) | 0.500 | (0.120) | 0.499 | (0.107) |
| FIGS. 7G-7I | 0.486 | (0.283) | 0.468 | (0.193) | 0.469 | (0.158) | 0.471 | (0.136) | 0.473 | (0.122) |
| FIGS. 7J-7K | 0.503 | (0.249) | 0.496 | (0.173) | 0.498 | (0.143) | 0.498 | (0.124) | 0.497 | (0.110) |
| Binary | 0.588 | (0.529) | 0.514 | (0.308) | 0.501 | (0.232) | 0.498 | (0.191) | 0.498 | (0.166) |

TABLE 4
Confidence metrics for human and simulated data

| | | | | | | | | | Deviance | | Deviance | |
|---|---|---|---|---|---|---|---|---|---|---|---|---|
| | BS | | REL | | RES | | UNC | | Binary | | CSD | |
| Subject 1 | 0.299 | (0.030) | 0.162 | (0.025) | 0.361 | (0.031) | 0.498 | (0.003) | 92.6 | (8.7) | 789.0 | (40.8) |
| Subject 2 | 0.254 | (0.043) | 0.147 | (0.034) | 0.384 | (0.033) | 0.491 | (0.007) | 75.3 | (10.1) | 752.6 | (44.8) |
| Subject 3 | 0.290 | (0.039) | 0.161 | (0.022) | 0.369 | (0.037) | 0.498 | (0.002) | 93.6 | (9.5) | 791.3 | (31.0) |
| Subject 4 | 0.293 | (0.049) | 0.152 | (0.029) | 0.352 | (0.023) | 0.493 | (0.008) | 79.2 | (12.8) | 821.0 | (20.6) |
| FIGS. 7A-7C | 0.270 | (0.038) | 0.190 | (0.029) | 0.415 | (0.030) | 0.495 | (0.007) | 79.3 | (8.0) | 764.6 | (37.4) |
| FIGS. 7D-7F | 0.282 | (0.030) | 0.183 | (0.025) | 0.396 | (0.032) | 0.495 | (0.007) | 79.3 | (8.0) | 807.9 | (18.2) |
| FIGS. 7G-7I | 0.304 | (0.029) | 0.185 | (0.026) | 0.376 | (0.034) | 0.495 | (0.007) | 79.3 | (8.0) | 830.7 | (22.7) |
| FIGS. 7J-7K | 0.300 | (0.028) | 0.185 | (0.025) | 0.380 | (0.033) | 0.495 | (0.007) | 79.3 | (8.0) | 788.0 | (21.7) |

Brier Score (BS) and three decomposed components; Reliability (REL), Resolution (RES), and Uncertainty (UNC) are shown. For definitions and descriptions, see Yates J F. Judgment and decision making. Prentice-Hall, Inc, 1990. We use a formulation that results by dividing Brier's original formulation by two. Deviance for each of two fits are also shown. Mean (and standard deviation) across 6 trials for each subject and across 10,000 simulations for each simulated data set are provided.

The simulated data (see, e.g., FIGS. 7A-7C, 1^{st} column of FIG. 8 corresponding to Panels A and E, and 2^{nd} row of Tables 2-4) show that the CSD model yielded fit parameters that are similar to those simulated when the simulated subject's confidence was well calibrated (k=1). A “well calibrated” subject, for example, has a confidence that is similar to the confidence represented by the psychometric function, χ(x)=ϕ(x;μ=0.5, kσ=1). Even when the subject's confidence was not well calibrated (k=2), the confidence fit parameters matched the three confidence function parameters well (see, e.g., FIGS. 7D-7F, 2^{nd} column of FIG. 8 corresponding to Panels B and F, and 3^{rd} row of Tables 2-4). In fact, except that the fitted confidence-scaling factor (\hat{k}) settles near a value of 2 (see, e.g., FIG. 7F) instead of 1 (see, e.g., FIG. 7E), the average psychometric parameter estimates for an under-confident subject appeared similar to the parameters for a well-calibrated subject. The fitted psychometric width parameter (\hat{σ}) demonstrated a lower standard deviation for an under-confident subject than for a well-calibrated subject (see FIGS. 9 and 10A-10D).

To demonstrate robustness, we utilized the same Gaussian confidence fit model (Equation 2) while simulating a confidence model that differed from the Gaussian confidence fit model. For example, we modeled the confidence function as a linear function (slope of 0.1445; i.e., σ=2) instead of a cumulative Gaussian. We also added zero-mean uniform noise, U(−0.1, 0.1), to the simulated confidence response. The confidence fit of these simulated data are similar to the earlier confidence fits well (see, e.g., FIGS. 7G-7I, and 3^{rd} column of FIG. 8 corresponding to Panels C and G, and 4^{th} row of Tables 2-4). The parameter fit precision was lower than the parameter fit precision for the first two simulation sets described above but was still higher than for the fits that do not use confidence techniques. For example, despite the severe noise (−10% to +10%), the fit precision for the width parameter (\hat{σ}) after twenty trials utilizing confidence matched the fit precision after about fifty trials using confidence-agnostic analyses.

Finally, to demonstrate the flexibility of the confidence fit technique, we model the same linear confidence function from the previous paragraph, but we now add less extreme zero-mean uniform noise levels (U(−0.05, 0.05)) and fit a linear confidence function that mimics the linearity of the true confidence function used for these simulations. The fit accuracy and precision were very good (FIGS. 7J-7L and the fourth column of FIG. 8 and 5^{th} row of Tables 2-4) demonstrating that the fitted psychometric function and confidence function need not be similar in form. Table 5 presents confidence metrics, including goodness of fit parameters.

### DISCUSSION

This disclosure describes a new confidence signal detection (CSD) model (FIGS. 1A-1D) and then uses this model to develop a confidence analysis technique (FIGS. 2A and 2B) that utilizes confidence probability judgments that can yield psychometric function fits using fewer experimental trials. The confidence analysis technique uses a confidence function-alongside confidence probability judgments—to improve psychometric function fit efficiency, thereby reducing the number of trials to generate the fit. The introduction of a fitted confidence function can yield several benefits. For example, it can incorporate confidence probability judgments into a psychometric fit procedure. In addition, while it does not require that the confidence function differ from the psychometric function, it allows these two functions to differ from one another.

We assumed that the binary decision variable is used to determine confidence probability judgments. Empirical data presented herein suggests that, at least for yaw rotation thresholds, confidence can correlate with the sampled decision variable used to make a decision.

Similar benefits can also result from replacing the confidence function and confidence recordings with a magnitude estimation function accompanied by magnitude estimation recordings (e.g., I rotated +3°). Other analogous perceptual functions and associated recordings could be used. The confidence probability judgments could alternatively or additionally be replaced by another confidence assay accompanied by an appropriate model of the confidence assay.

In this disclosure, we describe improving the efficiency of psychometric parameter fits. The fitting technique can also be used to calculate a confidence-scaling factor, which may be used in experimental studies examining confidence calibration. The confidence modeling approach described herein with respect to, for example, FIGS. 1A-1D, may be applied to studies examining confidence calibration.

### Human Studies

Human experimental data showed that stable psychometric function parameters could be estimated in as few as twenty trials, as shown in and described with respect to FIGS. 4A to 4L and 5. These data demonstrated no significant differences between the psychometric function parameters estimated after twenty trials using confidence data and the same parameters estimated after a hundred trials using confidence-agnostic methods. These findings suggest that the number of trials required to estimate psychometric fit parameters could be reduced by roughly 80% by collecting a confidence probability judgments and utilizing the confidence information in the manner described herein.

The four subjects had not previously performed experiments utilizing a confidence task prior to our studies of confidence. None of the subjects was provided feedback, except during the initial practice trials that consisted of 10 trials when subjects were informed whether their responses were correct or incorrect. No specific feedback regarding confidence was provided during the course of the study. Despite intentionally limiting the training and feedback, each subject yielded a coherent data set across the six test sessions. All four subjects were experienced observers.

For our task, we minimized the potential impact of after-effects from the previous trial by waiting at least 3 seconds between the end of a stimulus and the start of the next stimulus. Therefore, providing confidence did not require substantial additional time (e.g., greater than 3 seconds) to complete each trial than a confidence-agnostic binary forced-choice task for our direction-recognition task

For knowledge tasks, investigations showed that simple and short training sessions that can provide direct confidence calibration information can lead to improved calibration and that such improvement could generalize to tasks beyond the one calibrated. In some implementations, generalized calibration can be used to help calibrate confidence prior to testing as part of the process by which subjects are taught the requisite tasks. The generalized calibration techniques can be used during, for example, clinical testing. Alongside existing measures, such as, e.g., the Brier score and its calibration and resolution components, the confidence-scaling factor described herein could be used to determine if confidence calibration is an individual trait and to train confidence calibration. The confidence modeling approach can determine psychometric fit parameters in less than hundreds of trials. For example, the approach can determine the parameters in about twenty to fifty trials as shown and described with respect to FIGS. 4A to 4L and 5 to provide relatively stable calibration feedback. The calibration plots and the Brier score partitions used in previous studies can be sensitive to sample size.

### Simulations

Simulations confirmed that the confidence-based fitting technique described herein can yield accurate psychometric parameter estimates and a marked efficiency improvement. Simulations assuming (a) a confidence model that was not matched by the fitting model and (b) large additive confidence noise—likely greater than actual confidence noise—yielded psychometric function parameter estimates that were similar to the parameter estimates of the simulated psychometric function much more efficiently than confidence-agnostic psychometric fits (e.g., 20 trials as compared to 50-100 trials). Simulations thus indicate that the confidence signal detection methods can be robust to noise in the confidence function.

The confidence-based fitting method can be used for psychometric functions that range from, for example, 0 to 1, as confirmed by simulations. The fitting method can further be used for a specific vestibular direction-recognition task. Simulation results can be applicable to all tasks yielding psychometric functions that range from, for example, 0 to 1. Furthermore, the confidence-based technique can be applied to other tasks, such as, for example, detection tasks (e.g., yes/no tasks) or to two-alternative forced choice detection tasks where the subject identifies the interval (or location) when (or where) the signal occurred. These tasks can have different psychometric ranges (e.g., 0.5 to 1).

The CSD model depicted in, for example, FIGS. 1A-1D, can be used to develop a confidence analysis technique, as illustrated in FIGS. 2A and 2B, that utilizes confidence probability judgments to reduce the number of trials for psychometric parameter estimates. Human studies, example data from which is shown in FIGS. 4A to 4L and 5, using a direction-recognition task and a psychometric function that varies from 0 to 1 and simulations, whose example results are shown in FIGS. 6 to 8, suggest that about studies including about twenty trials using this new confidence fit method can match the performance achieved after about a hundred trials using confidence-agnostic fit methods.

### Computer System

FIG. 17 is a block diagram of a computing system 1700 at least a portion of which can be used for implementing the vestibular testing system 100.

The computing system 1700 can include, for example, a processor 1710, a memory 1720, a storage device 1730, and an input/output device 1740. Each of the components 1710, 1720, 1730, and 1740 are interconnected using a system bus 1750. The processor 1710 is capable of processing instructions for execution within the system 1700. In one implementation, the processor 1710 is a single-threaded processor. In another implementation, the processor 1710 is a multi-threaded processor. The processor 1710 is capable of processing instructions stored in the memory 1720 or on the storage device 1730 to display graphical information for a user interface on the input/output device 1740. The processor 1710 can be operable with electrical and electromechanical components of the vestibular testing system.

The memory 1720 stores information within the system 1700. In some implementations, the memory 1720 is a non-transitory computer-readable medium. The memory 1720 can include volatile memory and/or non-volatile memory.

The storage device 1730 is capable of providing mass storage for the system 1700. In one implementation, the storage device 1730 is a non-transitory computer-readable medium. In various different implementations, the storage device 1730 may be a hard disk device, an optical disk device, or a solid state memory device. The memory 1720 and/or the storage device 1730 can store treatment parameters and parameters of the electromechanical systems of the vestibular testing system described herein. These components can also store data collected by various sensors of the vestibular testing system. The memory 1720 and/or the storage device 1730 can also store data regarding the inputs (e.g., power input) into electromechanical components of the vestibular testing system. In some cases, the memory 1720 and/or the storage device 1730 can also store data pertaining to the progress of the treatment, such as the amount of fluid delivered or the duration of treatment that has elapsed.

The input/output device 1740 provides input/output operations for the system 1700. In some implementations, the input/output device 1740 includes a keyboard and/or a pointing device. In some implementations, the input/output device 1740 includes a display unit (e.g., a touchscreen display) for displaying graphical user interfaces. In some implementations the input/output device can be configured to accept verbal (e.g., spoken) inputs. The touchscreen display device may be, for example, a capacitive display device operable by touch, or a display that is configured to accept inputs via a stylus.

The features computing systems described herein can be implemented in digital electronic circuitry, or in computer hardware, firmware, or in combinations of these. The features can be implemented in a computer program product tangibly embodied in an information carrier, e.g., in a machine-readable storage device, for execution by a programmable processor; and features can be performed by a programmable processor executing a program of instructions to perform functions of the described implementations by operating on input data and generating output. The described features can be implemented in one or more computer programs that are executable on a programmable system including at least one programmable processor coupled to receive data and instructions from, and to transmit data and instructions to, a data storage system, at least one input device, and at least one output device. A computer program includes a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. Computers include a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

The processor 1710 carries out instructions related to a computer program. The processor 1710 may include hardware such as logic gates, adders, multipliers and counters. The processor 1710 may further include a separate arithmetic logic unit (ALU) that performs arithmetic and logical operations.

### OTHER EMBODIMENTS

It is to be understood that while the technology has been described in conjunction with the detailed description, the foregoing description and Examples are intended to illustrate and not limit the scope defined by the appended claims. Other aspects, advantages, and modifications are within the scope of the following claims.

What is claimed is:

1. An apparatus for estimating a vestibular function of a subject, the apparatus comprising:
a motion platform for supporting a subject, wherein the motion platform is configured to execute one or more motions;
an input device configured to receive confidence ratings from the subject, wherein the confidence ratings are related to the subject's perceptions of the one or more motions; and
one or more processing devices configured to:
fit a cumulative distribution function to the confidence ratings,
determine a relationship configured to link the cumulative distribution function to an underlying noise distribution, and
output a plurality of parameters associated with the vestibular function based at least in part on the relationship, wherein the plurality of parameters provides an estimate of the vestibular function of the subject.

2. The apparatus of claim 1, wherein the relationship is represented by a scaling parameter configured to link the cumulative distribution function associated with the confidence ratings to a cumulative distribution function associated with the underlying noise distribution.

3. The apparatus of claim 2, wherein the plurality of parameters includes the scaling parameter.

4. The apparatus of claim 2, wherein the cumulative distribution function associated with the confidence ratings and the cumulative distribution function associated with the underlying noise distribution are both Gaussian.

5. The apparatus of claim 1, wherein the plurality of parameters includes a width and bias of the vestibular function.

6. The apparatus of claim 1, wherein the confidence ratings comprise any of a quasi-continuous rating, a binary rating, a N-level discrete rating, or a wagering rating.

7. The apparatus of claim 1, wherein the cumulative distribution function is fitted to the confidence ratings using a maximum likelihood criterion.

8. A method for estimating a vestibular function of a subject, the method comprising:
providing, using a motion platform, one or more motion stimuli to a subject;
receiving, using an input device, confidence ratings from the subject, wherein the confidence ratings indicate the subject's perceptions of the motion stimuli;
fitting, by one or more processing devices, a cumulative distribution function to the confidence ratings;
determining, by the one or more processing devices, a relationship configured to link the cumulative distributive function to an underlying noise distribution; and
generating a plurality of parameters associated with the vestibular function based at least in part on the relationship, and wherein the plurality of parameters provides an estimation of the vestibular function of the subject.

9. The method of claim 8, wherein the relationship is represented by a scaling parameter configured to link the cumulative distribution function associated with the confidence ratings to a cumulative distribution function associated with the underlying noise distribution.

10. The method of claim 9, wherein the plurality of parameters includes the scaling parameter.

11. The method of claim 9, wherein the cumulative distribution function associated with the confidence ratings and the cumulative distribution function associated with the underlying noise distribution are both Gaussian.

12. The method of claim 8, wherein the plurality of parameters includes a width and bias of the vestibular function.

13. The method of claim 8, wherein the confidence ratings comprise any of a quasi-continuous rating, a binary rating, a N-level discrete rating, or a wagering rating.

14. The method of claim 8, wherein the cumulative distribution function is fitted to the confidence ratings using a maximum likelihood criterion.

15. One or more machine-readable storage devices having encoded thereon computer readable instructions for causing one or more processors to perform operations comprising:
providing one or more motion stimuli to a subject;
receiving confidence ratings from the subject, wherein the confidence ratings indicate the subject's perceptions of the motion stimuli;
fitting a cumulative distribution function to the confidence ratings;
determining a relationship configured to link the cumulative distributive function to an underlying noise distribution; and
generating a plurality of parameters associated with a vestibular function of the subject based at least in part on the relationship, and wherein the plurality of parameters provides an estimation of the vestibular function.

16. The one or more machine-readable storage devices of claim 15, wherein the relationship comprises a scaling parameter configured to link the cumulative distribution function associated with the confidence ratings to a cumulative distribution function associated with the underlying noise distribution.

17. The one or more machine-readable storage devices of claim 16, wherein the plurality of parameters includes the scaling parameter.

18. The one or more machine-readable storage devices of claim 16, wherein the cumulative distribution function associated with the confidence ratings and the cumulative distribution function associated with the underlying noise distribution are both Gaussian.

19. The one or more machine-readable storage devices of claim 15, wherein the plurality of parameters includes a width and bias of the vestibular function.

20. The one or more machine-readable storage devices of claim 15, wherein the confidence ratings comprise any of a quasi-continuous rating, a binary rating, a N-level discrete rating, or a wagering rating.
